(19) United States
(12) Patent Application Publication
(10) Pub. No.: US 2026/0234250 A1
(43) Pub. Date: Aug. 13, 2026

(54) COMPOSITIONS OF BUDOPRUTUG

(71) Applicant: Climb Bio Operating Inc., Wellesley, MA (US)

(72) Inventor: Tatyana Touzova, Scotch Plains, NJ (US)

(21) Appl. No.: 19/355,445

(22) Filed: Oct. 10, 2025

Related U.S. Application Data
(60) Provisional application No. 63/706,972, filed on Oct. 14, 2024.

(51) Int. Cl. C07K 16/28; A61K 47/02; A61K 47/12; A61K 47/22; A61K 47/26

(52) U.S. Cl. CPC C07K 16/2803; A61K 47/02; A61K 47/12; A61K 47/22; A61K 47/26

(57) ABSTRACT

The invention provides novel pharmaceutical compositions comprising anti-CD19 antibodies. The compositions of the invention are shelf-stable and suitable for administration to the patients.

### I. FIELD OF THE INVENTION

The invention is directed to a pharmaceutical composition comprising a therapeutically effective amount of an anti-CD19 antibody.

### II. SEQUENCE LISTING

The present application is being filed along with a Sequence Listing XML in electronic format. The Sequence Listing XML is provided as a file named CLIMB-008-01US-Seq-Listing.xml, created on Oct. 9, 2025, which is 55,918 bytes in size. The Sequence Listing XML file and the information in the electronic format of the Sequence Listing XML file is incorporated herein by reference in its entirety.

### III. BACKGROUND

CD19 is a surface protein found on B cells and on certain cancerous cells derived from B cells, such as many B cell lymphomas. Anti-CD19 monoclonal antibodies have been generated in mice, and show some promise in pre-clinical animal models of B cell-derived cancers. However, mouse-derived antibodies are generally immunogenic in humans. A number of strategies have been developed to alter mouse-derived antibodies to minimize their immunogenicity in humans. One such strategy, chimerization, involves the fusion of mouse variable regions to human constant regions. However, the mouse-derived variable region sequences remaining following chimerization will often be immunogenic. Another such strategy, humanization, involves the replacement of mouse-derived framework regions (FRs) within the variable regions with the most closely related human-derived sequences, with the optional reversion of certain amino acids back to the corresponding mouse amino acid in order to maintain binding activity. However, even humanized antibodies may be immunogenic, since the antibody complementarity determining regions (CDRs) generally contain B cell epitopes and T cell epitopes that are non-self. Indeed, even fully human antibodies are immunogenic; this is the basis for the formation of anti-idiotype antibodies during the course of an immune response. All of these problems may apply to mouse-derived anti-CD19 antibodies as they would to any other type of antibody. Therefore, there is a need for anti-CD19 antibodies with reduced immunogenicity.

### IV. SUMMARY OF THE INVENTION

The invention provides novel compositions comprising a therapeutically effective amount of an anti-CD19 antibody. In certain embodiments, the anti-CD19 antibody comprises: a heavy chain variable region at least 95% identical to SEQ ID NO: 17 and/or a light chain variable region at least 95% identical to SEQ ID NO: 29. In certain other embodiments, the anti-CD19 antibody is any anti-CD19 antibody provided herein.

Beneficially, the invention provides compositions comprising anti-CD19 antibodies, wherein the concentration of the anti-CD19 antibody is greater than 100 mg/mL. The compositions of the invention are shelf-stable, which is unexpected given the high concentration of anti-CD19 antibodies in the composition.

In certain aspects, the invention provides that the compositions of the invention may be administered subcutaneously. In certain aspects, the subcutaneous injection is self-administered by the patient. The self-administration could be upon direction of the physician. Self-administered subcutaneous injections can have several advantages, including: improved compliance, reduced healthcare costs, sense of autonomy, and an improved quality of life for the patients.

However, subcutaneous administration of compositions comprising a therapeutically effective amount of anti-CD19 antibodies has been challenging. In particular, the composition should be shelf-stable, should have a low volume, and have appropriate tonicity such that it may be administered through an injection of an appropriate size. Because anti-CD19 antibodies are large molecules, the development of compositions that fit the criteria for subcutaneous administration of such compositions has remained elusive. Indeed, the early-stage investigations with budoprutug, an anti-CD19 antibody, have all been undertaken as an intravenous (IV) infusion. However, IV infusions do not offer the advantages available with the subcutaneous administration of the solutions. Thus, there is an unmet need for development of compositions comprising an anti-CD19 antibody suitable for subcutaneous administration.

Beneficially, the compositions of the invention have an appropriate stability and tonicity such that they can be conveniently administered through an injection. In certain embodiments, the needle for the injection is a 27 G needle. It is desirable to have a composition that can be administered with a 27 G needle because of its small size it leads to less pain and chances of injury during administration of the composition. However, it is challenging to prepare a composition comprising a high concentration of an anti-CD19 antibodies that have the appropriate stability and tonicity for administration with a 27 G needle. The compositions of the invention may be administered through a 27 G needle.

In certain aspects, the invention provides a composition comprising a therapeutically effective amount of an anti-CD19 antibody. In certain embodiments, the anti-CD19 antibody comprises: a heavy chain variable region at least 95% identical to SEQ ID NO: 17 and/or a light chain variable region at least 95% identical to SEQ ID NO: 29. In certain embodiments, the anti-CD19 antibody comprises: a heavy chain variable region comprising SEQ ID NO: 17 and/or a light chain variable region comprising SEQ ID NO: 29. In certain embodiments, the composition comprises at least about 100 mg/mL of the anti-CD19 antibody.

In certain embodiments, the compositions of the invention comprises from about 100 mg/mL to about 300 mg/mL of the anti-CD19 antibody. In certain embodiments, the compositions of the invention comprises from about 100 mg/mL to about 300 mg/mL of budoprutug. In certain embodiments, the compositions of the invention comprises from about 180 mg/mL to about 220 mg/mL of budoprutug. In certain embodiments, the compositions of the invention comprises about 100 mg/mL of the anti-CD19 antibody. In certain embodiments, the compositions of the invention comprises about 100 mg/mL of budoprutug. In certain embodiments, the compositions of the invention comprises about 150 mg/mL of the anti-CD19 antibody. In certain embodiments, the compositions of the invention comprises about 150 mg/mL of budoprutug. In certain embodiments, the compositions of the invention comprises about 175 mg/mL of budoprutug. In certain embodiments, the compositions of the invention comprises about 180 mg/mL of budoprutug. In certain embodiments, the compositions of the invention comprises about 200 mg/mL of the anti-CD19 antibody. In certain embodiments, the compositions of the invention comprises about 200 mg/mL of budoprutug. In certain embodiments, the compositions of the invention comprises about 220 mg/mL of budoprutug.

In certain embodiments, the compositions of the invention comprise a pharmaceutically acceptable excipient. In certain embodiments, the compositions of the invention comprise a buffer. In certain embodiments, the buffer is a pharmaceutically acceptable buffer. In certain embodiments, the buffer is selected from the group consisting of citrate, acetate, tromethamine, phosphate, hydroxymethylaminoethane, histidine, and glutamate. In certain preferred embodiments, the buffer is citrate. In certain preferred embodiments, the buffer is histidine.

In certain embodiments, the compositions of the invention comprises about 10 mM to about 100 mM of the pharmaceutically acceptable buffer. In certain embodiments, the compositions of the invention comprises about 10 mM to about 50 mM of the pharmaceutically acceptable buffer. In certain embodiments, the compositions of the invention comprises about 5 mM of the pharmaceutically acceptable buffer. In certain embodiments, the compositions of the invention comprises about 10 mM of the pharmaceutically acceptable buffer. In certain embodiments, the compositions of the invention comprises about 15 mM of the pharmaceutically acceptable buffer. In certain embodiments, the compositions of the invention comprises about 20 mM of the pharmaceutically acceptable buffer. In certain embodiments, the compositions of the invention comprises about 25 mM of the pharmaceutically acceptable buffer.

In certain embodiments, the pH of the compositions of the invention is from about 4 to about 8. In certain embodiments, the pH of the compositions of the invention is from about 5 to about 7. In certain embodiments, the pH of the compositions of the invention is about 5. In certain embodiments, the pH of the compositions of the invention is about 5.5. In certain embodiments, the pH of the compositions of the invention is about 6. In certain embodiments, the pH of the compositions of the invention is about 6.5. In certain embodiments, the pH of the compositions of the invention is about 7. In certain embodiments, the pH of the compositions of the invention is about 7.5.

In certain embodiments, the compositions of the invention further comprises a tonicity modifier. The tonicity modifier is an important excipient in the composition of the invention. In particular, the tonicity modifier impacts the viscosity of the pharmaceutical composition. Thus, tonicity modifier plays a role in ensuring that the composition may be administered via subcutaneous injection. In certain embodiments, the tonicity modifier is selected from the group consisting of sodium chloride, arginine, sorbitol, dextrose, mannitol, potassium chloride, maltose, trehalose, glycerol, or any combination thereof. In certain embodiments, the tonicity modifier is selected from the group consisting of sodium chloride, arginine, sorbitol, and any combination thereof. In certain embodiments, the tonicity modifier is arginine.

In certain embodiments, the composition comprises from about 50 mM to about 200 mM of the tonicity modifier. In certain embodiments, the composition comprises from about 50 mM to about 150 mM of the tonicity modifier. In certain embodiments, the composition comprises from about 50 mM of the tonicity modifier. In certain embodiments, the composition comprises from about 100 mM of the tonicity modifier. In certain embodiments, the composition comprises from about 150 mM of the tonicity modifier. In certain embodiments, the composition comprises from about 200 mM of the tonicity modifier. In certain embodiments, the composition comprises from about 1% to about 10% w/v of the tonicity modifier. In certain embodiments, the composition comprises from about 2.5% to about 10% w/v of the tonicity modifier. In certain embodiments, the composition comprises from about 5% to about 10% w/v of the tonicity modifier. In certain embodiments, the composition comprises from about 5% w/v of the tonicity modifier. In certain embodiments, the composition comprises from about 7.5% w/v of the tonicity modifier. In certain embodiments, the composition comprises from about 10% w/v of the tonicity modifier.

In certain embodiments, the viscosity of the pharmaceutical composition of the invention is less than 50 cP. In certain embodiments, the viscosity of the pharmaceutical composition of the invention is less than 40 cP. In certain embodiments, the viscosity of the pharmaceutical composition of the invention is less than 35 cP. In certain preferred embodiments, the viscosity of the pharmaceutical composition of the invention is less than 30 cP. In certain preferred embodiments, the viscosity of the pharmaceutical composition of the invention is less than 25 cP. In certain preferred embodiments, the viscosity of the pharmaceutical composition of the invention is less than 20 cP. In certain preferred embodiments, the viscosity of the pharmaceutical composition of the invention is less than 15 cP.

In certain embodiments, the composition comprises a surfactant. The surfactant could be any pharmaceutically acceptable excipient. In certain embodiments, the surfactant is a polysorbate. In certain embodiments, the surfactant is polysorbate 80.

### V. BRIEF DESCRIPTION OF THE DRAWINGS

FIG. 1 provides the pictures of samples after agitation study.

FIG. 2 provides the results for the HPLC analysis from the agitation study.

FIG. 3 provides the pictures of samples after light stability study.

FIG. 4 provides the results for the HPLC analysis from the light stability study.

FIG. 5 provides exemplary formulations with a concentration screen.

FIG. 6 provides the pictures of samples after the concentration feasibility study.

FIG. 7 provides the results for the HPLC analysis from the concentration feasibility study.

FIG. 8 provides the pictures for samples after the thermal stability studies for 25° C., and 40° C. at 2 weeks.

FIG. 9 provides the results from visual appearance from the T4 weeks study for samples stored at 2-8° C., 25° C., and 40° C.

FIG. 10 provides the results from visual appearance from the T8 weeks study for samples stored at 2-8° C., 25° C., and 40° C.

### VI. DETAILED DESCRIPTION

The following is a detailed definition of the terms used in the specification.

The term “shelf-stable pharmaceutical composition” as used herein means a pharmaceutical composition which is stable for at least the period which is required by regulatory agencies in connection with therapeutic proteins. Preferably, a shelf-stable pharmaceutical composition is stable for at least one year at 5° C. Stability includes chemical stability as well as physical stability.

The term “effective amount” as used herein means a dosage which is sufficient in order for the treatment of the patient to be effective compared with no treatment.

The term “composition” or “pharmaceutical composition” as used herein means a product comprising an active compound or a salt thereof together with pharmaceutical excipients such as buffer, preservative and tonicity modifier, said pharmaceutical composition being useful for treating, preventing or reducing the severity of a disease or disorder by administration of said pharmaceutical composition to a person. Thus a pharmaceutical composition is also known in the art as a pharmaceutical formulation. It is to be understood that pH of a pharmaceutical composition which is to be reconstituted is the pH value which is measured on the reconstituted composition produced by reconstitution in the prescribed reconstitution liquid at room temperature.

The term “pharmaceutically acceptable” as used herein means suited for normal pharmaceutical applications, i.e. giving rise to no adverse events in patients etc.

The term “buffer” as used herein refers to a chemical compound in a pharmaceutical composition that reduces the tendency of pH of the composition to change over time as would otherwise occur due to chemical reactions. Buffers include chemicals such as sodium phosphate, TRIS, glycine and sodium citrate.

The term “preservative” as used herein refers to a chemical compound which is added to a pharmaceutical composition to prevent or delay microbial activity (growth and metabolism). Examples of pharmaceutically acceptable preservatives are phenol, m-cresol and a mixture of phenol and m-cresol.

The term “tonicity modifier” as used refers to a chemical compound in a pharmaceutical composition that serves to modify the osmotic pressure of the pharmaceutical composition so that the osmotic pressure becomes closer to that of human plasma.

The term “stabilizer” as used herein refers to chemicals added to peptide containing pharmaceutical compositions in order to stabilize the peptide, i.e. to increase the shelf life and/or in-use time of such compositions. Examples of stabilizers used in pharmaceutical formulations are L-glycine, L-histidine, arginine, polyethylene glycol, and carboxymethylcellulose.

The term “Surfactant” as used herein refers to any molecules or ions that are comprised of a water-soluble (hydrophilic) part, the head, and a fat-soluble (lipophilic) segment. Surfactants accumulate preferably at interfaces, which the hydrophilic part is orientated towards the water (hydrophilic phase) and the lipophilic part towards the oil- or hydrophobic phase (i.e. glass, air, oil etc.). The concentration at which surfactants begin to form micelles is known as the critical micelle concentration or CMC. Furthermore, surfactants lower the surface tension of a liquid. Surfactants are also known as amphipathic compounds. The term “Detergent” is a synonym used for surfactants in general.

### Anti-CD19 Antibodies of the Invention:

In certain aspects, the invention provides anti-CD19 antibodies which feature an amino acid sequence defining a modified immunoglobulin heavy chain framework region comprising amino acid residues 1-30 of SEQ ID NO:22, wherein one or more of the amino acid residues at positions X5, X12, X19, X20, X23, and X24 are as follows: X5 is Q or E, X12 is V or K, X19 is R or K, X20 is L or V, X23 is K, E or D, or X24 is T or A. In certain embodiments, at least one of the amino acid residues at positions X5, X12, X19, X20, X23, or X24 is not the same amino acid residue as the amino acid at the corresponding position in the unmodified immunoglobulin heavy chain framework region as set forth in amino acid residues 1-30 of SEQ ID NO:13. In one embodiment, X23 is E or D.

In certain embodiments, the anti-CD19 antibodies feature an amino acid sequence defining a modified immunoglobulin heavy chain framework region comprising amino acid residues 1-14 of SEQ ID NO:23, wherein one or more of the amino acid residues at positions X3, X5, X7, and X8, are as follows: X3 is K or R, X5 is R, T, or A, X7 is G, D, or E, or X8 is Q or K. According to this aspect of the invention, at least one of the amino acid residues at positions X3, X5, X7, or X8 is not the same as the amino acid at the corresponding position in the unmodified immunoglobulin heavy chain framework region as set forth in amino acid residues 36-49 of SEQ ID NO:13. In one embodiment, X7 is E or D.

In certain embodiments, the anti-CD19 antibodies feature an amino acid sequence defining a modified immunoglobulin heavy chain framework region comprising amino acid residues 1-39 of SEQ ID NO:24, wherein one or more of the amino acid residues at positions X6, X10, X26, X29, and X34 are as follows: X6 is K, D, or E, X10 is K, E, or D, X26 is S, D, or E, X29 is S or A, or X34 is V or T. According to this aspect of the invention, at least one of the amino acid residues at positions X6, X10, X26, X29, or X34 is not the same as the amino acid at the corresponding position in the unmodified immunoglobulin heavy chain framework region as set forth in amino acid residues 60-98 of SEQ ID NO: 13. In one embodiment, X10 is E or D.

In certain embodiments, the anti-CD19 antibodies feature an amino acid sequence defining a modified immunoglobulin light chain framework region comprising amino acid residues 1-23 of SEQ ID NO:32, wherein one or more of the amino acid residues at positions X1, X3, X7, X10, X11, and X19 are as follows: X1 is Q or D, X3 is V or A, X7 is S or E, X10 is I or T, X11 is M or L, or X19 is V or A. According to this aspect of the invention, at least one of the amino acid residues at positions X1, X3, X7, X10, X11, or X19 is not the same as the amino acid at the corresponding position in the unmodified immunoglobulin light chain framework region as set forth in amino acid residues 1-23 of SEQ ID NO:25. In one embodiment, X3 is A and X7 is E. In another embodiment, X1 is D, X10 is I, and X11 is L.

In certain embodiments, the invention features an amino acid sequence defining a modified immunoglobulin light chain complementarity determining region comprising amino acid residues 24-33 of SEQ ID NO:28.

In certain embodiments, the invention features an amino acid sequence defining a modified immunoglobulin light chain framework region comprising amino acid residues 56-87 of SEQ ID NO: 28.

According to another aspect, the invention features an antibody variable region comprising an amino acid sequence selected from the group consisting of SEQ ID NO: 14, SEQ ID NO: 15, SEQ ID NO: 16, SEQ ID NO: 17, SEQ ID NO: 20, SEQ ID NO: 21, SEQ ID NO: 26, SEQ ID NO: 27, SEQ ID NO: 28, SEQ ID NO: 29, SEQ ID NO: 30, or SEQ ID NO: 31, wherein the antibody variable region specifically binds to CD19.

In certain embodiments, the invention features a polypeptide at least 90% or at least 95% identical to a B4 antibody heavy chain variable region, the polypeptide comprising an amino acid substitution at one or more residues corresponding to Val12, Leu20, Lys23, Thr24, Lys38, Gly42, Gln43, Lys65, Lys69, Ser85, Ser88, or Val93. In one embodiment, the polypeptide comprises one or more of substitutions Gln5Glu, Val12Lys, Arg19Lys, Leu20Val, Lys23Glu, Lys23 Asp, Thr24Ala, Lys38Arg, Arg40Thr, Gly42Asp, Gly42Glu, Gln43Lys, Lys65Asp, Lys65Glu, Lys69Glu, Lys69Asp, Ser85Asp, Ser85Glu, Ser88Ala, or Val93Thr.

According to another aspect, the invention features a polypeptide at least 90% or at least 95% identical to a B4 antibody light chain variable region, the polypeptide comprising an amino acid substitution at one or more residues corresponding to Val3, Ser7, Ile10, Met11, Val19, Val29, Ser51, Leu53, Ala54, or Ser75. In one embodiment, the polypeptide comprises one or more of substitutions Gln1Asp, Val3Ala, Ser7Glu, Ile10Thr, Met11Leu, Val19Ala, Val29Ala, Ser51Asp, Leu53Thr, Ala54Asp, or Ser75Glu.

In certain aspects, the invention provides a process for preparation of anti-CD19 antibody, wherein the anti-CD19 antibody comprises variable domain comprising a light chain variable region that is at least 90% identical to SEQ ID NO: 25 and has an amino acid substitution at one or more residues corresponding to Ile10, Met11, Val19, Ser51, and Leu53; and the process comprises production of the anti-CD19 antibody in Chinese Hamster Ovary (CHO) cells.

In certain aspects, the invention provides a process for preparation of an anti-CD19 antibody, wherein: the anti-CD19 antibody comprises SEQ ID NO: 13 with an amino acid substitution at one or more residues corresponding to Gln5, Arg19, Leu20, Arg40, Gln43, Lys65, Ser85, Ser88, and Val93, and SEQ ID NO: 25 with an amino acid substitution at one or more residues corresponding to Ile10, Met11, Val19, Ser51, and Leu53; and the process comprises production of the anti-CD19 antibody in Chinese Hamster Ovary (CHO) cells.

In certain embodiments, the anti-CD19 antibody variable domain comprises a heavy chain variable region of SEQ ID NO: 17 and a light chain variable region of SEQ ID NO: 29. In certain embodiments, the anti-CD19 antibody is provided in U.S. Pat. No. 8,691,952, which is incorporated by reference in its entirety. In certain embodiments, the anti-CD19 antibody variable domain comprises a heavy chain variable region of SEQ ID NO: 17 and a light chain variable region of SEQ ID NO: 29 are provided in U.S. Pat. No. 8,691,952.

In certain embodiments, the heavy chain variable region of the anti-CD19 antibody has, compared to SEQ ID NO:13, one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, the heavy chain variable region of the anti-CD19 antibody is SEQ ID NO: 13 comprising an amino acid substitution at one or more residues corresponding to Gln5, Arg19, Leu20, Arg40, Gln43, Lys65, Ser85, Ser88, and Val93.

In certain embodiments, the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody is the amino acid sequence of SEQ ID NO: 17.

In certain embodiments, the anti-CD19 antibody variable domain of the anti-CD19 antibody further comprises a light chain variable region that is at least 90% identical to SEQ ID NO: 25 and has an amino acid substitution at one or more residues corresponding to Ile10, Met11, Val19, Ser51, and Leu53.

In certain embodiments, wherein the light chain variable region the anti-CD19 antibody has one or more amino acid substitutions selected from the group consisting of Ile10Thr, Met11Leu, Val19Ala, Ser51Asp, and Leu53Thr.

In certain embodiments, the light chain variable region is at least 95% identical to SEQ ID NO: 25.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is SEQ ID NO: 25 comprising an amino acid substitution at one or more residues corresponding to Ile10, Met11, Val19, Ser51, and Leu53.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is the amino acid sequence of SEQ ID NO: 29.

In certain embodiments, the light chain variable region and heavy chain variable region of the anti-CD19 antibody comprise amino acid sequences recited in SEQ ID NO: 29 and SEQ ID NO: 17 respectively.

In certain embodiments, the heavy chain variable region comprises one or more of substitutions Gln5Glu, Arg19Lys, Leu20Val, Arg40Thr, Gln43Lys, Lys65Asp, Ser85Asp, Ser88Ala, and Val93Thr.

In certain embodiments, wherein the light chain variable region comprises one or more of substitutions Ile10Thr, Met11Leu, Val19Ala, Ser51Asp, and Leu53Thr.

In certain embodiments, the anti-CD19 antibody is VB119. In certain embodiments, VB119 is an anti-CD19, IgG1 monoclonal antibody. VB119 demonstrates antibody-dependent cell mediated toxicity (ADCC) with minimal to no complement dependent cytotoxicity (CDC).

In certain embodiments, the anti-CD19 antibody comprises one or more of the below listed subunits in the anti-CD19 antibody:

Subunit 1 (SEQ ID NO: 50):
QVQLEQPGAEVVKPGASVKVSCKTSGYTFTSNWMHWVKQTPGKGLEWIGE
IDPSDSYTNYNQKFDGKAKLTVDKSSSTAYMEVSDLTAEDSATYYCARGS
NPYYYAMDYWGQGTSVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
Subunit 2 (SEQ ID NO: 51):
QVQLEQPGAEVVKPGASVKVSCKTSGYTFTSNWMHWVKQTPGKGLEWIGE
IDPSDSYTNYNQKFDGKAKLTVDKSSSTAYMEVSDLTAEDSATYYCARGS
NPYYYAMDYWGQGTSVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
Subunit 3 (SEQ ID NO: 52):
QIVLTQSPATLSASPGEKATMTCSASSGVNYMHWYQQKPGTSPKRWIYDT
DKTASGVPARFSGSGSGTSYSLTISSMEAEDAATYYCHQRGSYTFGGGTK
LEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSS
PVTKSFNRGEC
Subunit 4 (SEQ ID NO: 53):
QIVLTQSPATLSASPGEKATMTCSASSGVNYMHWYQQKPGTSPKRWIYDT
DKTASGVPARFSGSGSGTSYSLTISSMEAEDAATYYCHQRGSYTFGGGTK
LEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSS
PVTKSFNRGEC
In certain embodiments, the heavy chain sequence
of the anti-CD19 antibody is provided below (SEQ
ID NO: 54):
QVQLEQPGAEVVKPGASVKVSCKTSGYTFTSNWMHWVKQTPGKGLEWIGE
IDPSDSYTNYNQKFDGKAKLTVDKSSSTAYMEVSDLTAEDSATYYCARGS
NPYYYAMDYWGQGTSVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
In certain embodiments, the light chain sequence
of the anti-CD19 antibody is provided below (SEQ
ID NO: 55):
QIVLTQSPATLSASPGEKATMTCSASSGVNYMHWYQQKPGTSPKRWIYDT
DKTASGVPARFSGSGSGTSYSLTISSMEAEDAATYYCHQRGSYTFGGGTK
LEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSS
PVTKSFNRGEC

In certain embodiments, the anti-CD19 antibody is budoprutug. In certain embodiments, budoprutug comprises a heavy chain and a light chain. In certain embodiments, budoprutug comprises heavy chain and light chain provided in SEQ ID NO: 54 and SEQ ID NO: 55 respectively.

In certain embodiments, the heavy chain of anti-CD19 antibody is at least 90% identical to SEQ ID NO: 54. In certain embodiments, the heavy chain of anti-CD19 antibody is at least 95% identical to SEQ ID NO: 54. In certain embodiments, the heavy chain of anti-CD19 antibody is at least 98% identical to SEQ ID NO: 54. In certain embodiments, the heavy chain of anti-CD19 antibody is identical to SEQ ID NO: 54.

In certain embodiments, the light chain of anti-CD19 antibody is at least 90% identical to SEQ ID NO: 55. In certain embodiments, the light chain of anti-CD19 antibody is at least 95% identical to SEQ ID NO: 55. In certain embodiments, the light chain of anti-CD19 antibody is at least 98% identical to SEQ ID NO: 55. In certain embodiments, the light chain of anti-CD19 antibody is identical to SEQ ID NO: 55.

### Fc Portion

In certain embodiments, the antibody variable domains of the antibodies prepared using the methods of the invention are optionally fused to an Fc portion. As used herein, the Fc portion encompasses domains derived from the heavy chain constant region of an immunoglobulin, preferably a human immunoglobulin, including a fragment, analog, variant, mutant or derivative of the constant region. The constant region of an immunoglobulin heavy chain is defined as a naturally-occurring or synthetically produced polypeptide homologous to at least a portion of the C-terminal region of the heavy chain, including the CH1, hinge, CH2, CH3, and, for some heavy chain classes, CH4 domains. The “hinge” region joins the CH1 domain to the CH2-CH3 region of an Fc portion. The constant region of the heavy chains of all mammalian immunoglobulins exhibit extensive amino acid sequence similarity.

In the present invention, the Fc portion typically includes at least a CH2 domain. For example, the Fc portion can include the entire immunoglobulin heavy chain constant region (CH1-hinge-CH2-CH3). Alternatively, the Fc portion can include all or a portion of the hinge region, the CH2 domain and the CH3 domain.

The constant region of an immunoglobulin is responsible for many important antibody effector functions, including those mediated by Fc receptor (FcR) binding and by complement binding. There are five major classes of the heavy chain constant region, classified as IgA, IgG, IgD, IgE, and IgM, each with characteristic effector functions designated by isotype.

IgG, for example, is separated into four γ isotypes: γ1, γ2, γ3, and γ4, also known as IgG1, IgG2, IgG3, and IgG4, respectively. IgG molecules can interact with multiple classes of cellular receptors including three classes of Fcγ receptors (FcγR) specific for the IgG class of antibody, namely FcγRI, FcγRII, and FcγRIII. The sequences important for the binding of IgG to the FcγR receptors have been reported to be in the CH2 and CH3 domains.

It is also often useful to alter the serum half-life of the antibody. The serum half-life of an antibody, as of an immunoglobulin fusion protein, is influenced by the ability of that antibody to bind to an Fc receptor (FcR) (Gillies et al., Cancer Research (1999) 59:2159-66). The CH2 and CH3 domains of IgG2 and IgG4 have undetectable or reduced binding affinity to Fc receptors compared to those of IgG1. Accordingly, the serum half-life of the featured antibody can be increased by using the CH2 and/or CH3 domain from IgG2 or IgG4 isotypes. Alternatively, the antibody can include a CH2 and/or CH3 domain from IgG1 or IgG3 with modification in one or more amino acids in these domains to reduce the binding affinity for Fc receptors (see, e.g., U.S. patent application Ser. No. 09/256,156, published as U.S. patent application publication 2003-0105294).

In certain embodiments, an Fc portion fused to an antibody variable region of the invention can contain CH2 and/or CH3 domains and a hinge region that are derived from different antibody isotypes. For example, the Fc portion can contain CH2 and/or CH3 domains of IgG2 or IgG4 and a hinge region of IgG1. Assembly of such hybrid Fc portions has been described in U.S. patent application publication 2003-0044423.

When fused to an antibody variable region of the invention, the Fc portion may contain one or more amino acid modifications that generally extend the serum half-life of an Fc fusion protein. Such amino acid modifications include mutations substantially decreasing or eliminating Fc receptor binding or complement fixing activity. For example, one type of such mutation removes the glycosylation site of the Fc portion of an immunoglobulin heavy chain. In IgG1, the glycosylation site is Asn297 (see, for example, U.S. patent application Ser. No. 10/310,719, published as U.S. patent application publication 2003-0166163).

### Compositions of the Invention

The invention provides novel compositions comprising a therapeutically effective amount of an anti-CD19 antibody. In certain embodiments, the anti-CD19 antibody comprises: a heavy chain variable region at least 95% identical to SEQ ID NO: 17 and/or a light chain variable region at least 95% identical to SEQ ID NO: 29. In certain other embodiments, the anti-CD19 antibody is any anti-CD19 antibody provided herein.

Beneficially, the invention provides compositions comprising anti-CD19 antibodies, wherein the concentration of the anti-CD19 antibody is greater than 100 mg/mL. The compositions of the invention are shelf-stable, which is unexpected given the high concentration of anti-CD19 antibodies in the composition.

In certain aspects, the invention provides that the compositions of the invention may be administered subcutaneously. In certain aspects, the subcutaneous injection is self-administered by the patient. The self-administration could be upon direction of the physician. Self-administered subcutaneous injections can have several advantages, including: improved compliance, reduced healthcare costs, sense of autonomy, and an improved quality of life for the patients.

However, subcutaneous administration of compositions comprising a therapeutically effective amount of anti-CD19 antibodies has been challenging. In particular, the composition should be shelf-stable, should have a low volume, and have appropriate tonicity such that it may be administered through an injection of an appropriate size. Because anti-CD19 antibodies are large molecules, the development of compositions that fit the criteria for subcutaneous administration of such compositions has remained elusive. Indeed, the early-stage investigations with budoprutug, an anti-CD19 antibody, have all been undertaken as an intravenous (IV) infusion. However, IV infusions do not offer the advantages available with the subcutaneous administration of the solutions. Thus, there is an unmet need for development of compositions comprising an anti-CD19 antibody suitable for subcutaneous administration.

Beneficially, the compositions of the invention have an appropriate stability and tonicity such that they can be conveniently administered through an injection. In certain embodiments, the needle for the injection is a 27 G needle. It is desirable to have a composition that can be administered with a 27 G needle because of its small size it leads to less pain and chances of injury during administration of the composition. However, it is challenging to prepare a composition comprising a high concentration of an anti-CD19 antibodies that have the appropriate stability and tonicity for administration with a 27 G needle. The compositions of the invention may be administered through a 27 G needle.

In certain aspects, the invention provides a composition comprising a therapeutically effective amount of an anti-CD19 antibody. In certain embodiments, the anti-CD19 antibody comprises: a heavy chain variable region at least 95% identical to SEQ ID NO: 17 and/or a light chain variable region at least 95% identical to SEQ ID NO: 29. In certain embodiments, the composition comprises at least about 100 mg/mL of the anti-CD19 antibody.

In certain embodiments, the compositions of the invention comprises from about 100 mg/mL to about 300 mg/mL of the anti-CD19 antibody. In certain embodiments, the compositions of the invention comprises from about 100 mg/mL to about 300 mg/mL of budoprutug. In certain embodiments, the compositions of the invention comprises from about 180 mg/mL to about 220 mg/mL of budoprutug. In certain embodiments, the compositions of the invention comprises about 100 mg/mL of the anti-CD19 antibody. In certain embodiments, the compositions of the invention comprises about 100 mg/mL of budoprutug. In certain embodiments, the compositions of the invention comprises about 150 mg/mL of the anti-CD19 antibody. In certain embodiments, the compositions of the invention comprises about 150 mg/mL of budoprutug. In certain embodiments, the compositions of the invention comprises about 175 mg/mL of budoprutug. In certain embodiments, the compositions of the invention comprises about 180 mg/mL of budoprutug. In certain embodiments, the compositions of the invention comprises about 200 mg/mL of the anti-CD19 antibody. In certain embodiments, the compositions of the invention comprises about 200 mg/mL of budoprutug. In certain embodiments, the compositions of the invention comprises about 220 mg/mL of budoprutug.

In certain embodiments, the compositions of the invention comprise a pharmaceutically acceptable excipient. In certain embodiments, the compositions of the invention comprise a buffer. In certain embodiments, the buffer is a pharmaceutically acceptable buffer. In certain embodiments, the buffer is selected from the group consisting of phosphate, glycyl-glycine, TRIS, bicine, HEPES, MOBS, MOPS, TES and mixtures thereof.

In certain embodiments, the buffer is selected from the group consisting of citrate, acetate, tromethamine, phosphate, hydroxymethylaminoethane, histidine, and glutamate. In certain preferred embodiments, the buffer is citrate. In certain preferred embodiments, the buffer is histidine.

In certain embodiments, the compositions of the invention comprises about 10 mM to about 100 mM of the pharmaceutically acceptable buffer. In certain embodiments, the compositions of the invention comprises about 10 mM to about 50 mM of the pharmaceutically acceptable buffer. In certain embodiments, the compositions of the invention comprises about 5 mM of the pharmaceutically acceptable buffer. In certain embodiments, the compositions of the invention comprises about 10 mM of the pharmaceutically acceptable buffer. In certain embodiments, the compositions of the invention comprises about 15 mM of the pharmaceutically acceptable buffer. In certain embodiments, the compositions of the invention comprises about 20 mM of the pharmaceutically acceptable buffer. In certain embodiments, the compositions of the invention comprises about 25 mM of the pharmaceutically acceptable buffer.

In certain embodiments, the pH of the compositions of the invention is from about 4 to about 8. In certain embodiments, the pH of the compositions of the invention is from about 5 to about 7. In certain embodiments, the pH of the compositions of the invention is about 5. In certain embodiments, the pH of the compositions of the invention is about 5.5. In certain embodiments, the pH of the compositions of the invention is about 6. In certain embodiments, the pH of the compositions of the invention is about 6.5. In certain embodiments, the pH of the compositions of the invention is about 7. In certain embodiments, the pH of the compositions of the invention is about 7.5.

In certain embodiments, the compositions of the invention further comprises a tonicity modifier. The tonicity modifier is an important excipient in the composition of the invention. In particular, the tonicity modifier impacts the viscosity of the pharmaceutical composition. Thus, tonicity modifier plays a role in ensuring that the composition may be administered via subcutaneous injection. In certain embodiments, the tonicity modifier is selected from the group consisting of sodium chloride, arginine, sorbitol, dextrose, mannitol, propylene glycol, potassium chloride, maltose, trehalose, glycerol, or any combination thereof. In certain embodiments, the tonicity modifier is selected from the group consisting of sodium chloride, arginine, sorbitol, and any combination thereof. In certain embodiments, the tonicity modifier is arginine.

In certain embodiments, the composition comprises from about 50 mM to about 200 mM of the tonicity modifier. In certain embodiments, the composition comprises from about 50 mM to about 150 mM of the tonicity modifier. In certain embodiments, the composition comprises from about 50 mM of the tonicity modifier. In certain embodiments, the composition comprises from about 100 mM of the tonicity modifier. In certain embodiments, the composition comprises from about 150 mM of the tonicity modifier. In certain embodiments, the composition comprises from about 200 mM of the tonicity modifier. In certain embodiments, the composition comprises from about 1% to about 10% w/v of the tonicity modifier. In certain embodiments, the composition comprises from about 2.5% to about 10% w/v of the tonicity modifier. In certain embodiments, the composition comprises from about 5% to about 10% w/v of the tonicity modifier. In certain embodiments, the composition comprises from about 5% w/v of the tonicity modifier. In certain embodiments, the composition comprises from about 7.5% w/v of the tonicity modifier. In certain embodiments, the composition comprises from about 10% w/v of the tonicity modifier.

In certain embodiments, the viscosity of the pharmaceutical composition of the invention is less than 50 cP. In certain embodiments, the viscosity of the pharmaceutical composition of the invention is less than 40 cP. In certain embodiments, the viscosity of the pharmaceutical composition of the invention is less than 35 cP. In certain preferred embodiments, the viscosity of the pharmaceutical composition of the invention is less than 30 cP. In certain preferred embodiments, the viscosity of the pharmaceutical composition of the invention is less than 25 cP. In certain preferred embodiments, the viscosity of the pharmaceutical composition of the invention is less than 20 cP. In certain preferred embodiments, the viscosity of the pharmaceutical composition of the invention is less than 15 cP.

In certain embodiments, the composition comprises a surfactant. The surfactant could be any pharmaceutically acceptable excipient. In certain embodiments, the surfactant is a polysorbate. In certain embodiments, the surfactant is polysorbate 80. In certain embodiments, the compositions of the invention comprise about 0.005%-0.1% w/v polysorbate 80. In certain embodiments, the compositions of the invention comprise about 0.01%-0.05% w/v polysorbate 80. In certain preferred embodiments, the compositions of the invention comprise about 0.02% w/v polysorbate 80 (PS80).

In certain embodiments, the invention provides that the compositions of the invention further comprise a preservative. In certain embodiments, the preservative is selected from the group consisting of phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, thimerosal and mixtures thereof.

### Exemplary Formulations

Exemplary formulations of the invention are provided in the table below.

| Formulation # | Buffer | pH | Tonicity modifier | Surfactant |
|---|---|---|---|---|
| #16 | 25 mM L- | 6 | 4% Sorbitol | PS80 0.02% (w/v) |
| | Histidine | | | |
| #11 | 25 mM | 6 | 50 mM Arginine, | PS80 0.02% (w/v) |
| | Citrate | | 50 mM NaCl | |
| #10 | 25 mM | 6 | 100 mM Arginine | PS80 0.02% (w/v) |
| | Citrate | | | |
| #9 | 25 mM | 6 | 150 mM NaCl | PS80 0.02% (w/v) |
| | Citrate | | | |

### Agitation Studies:

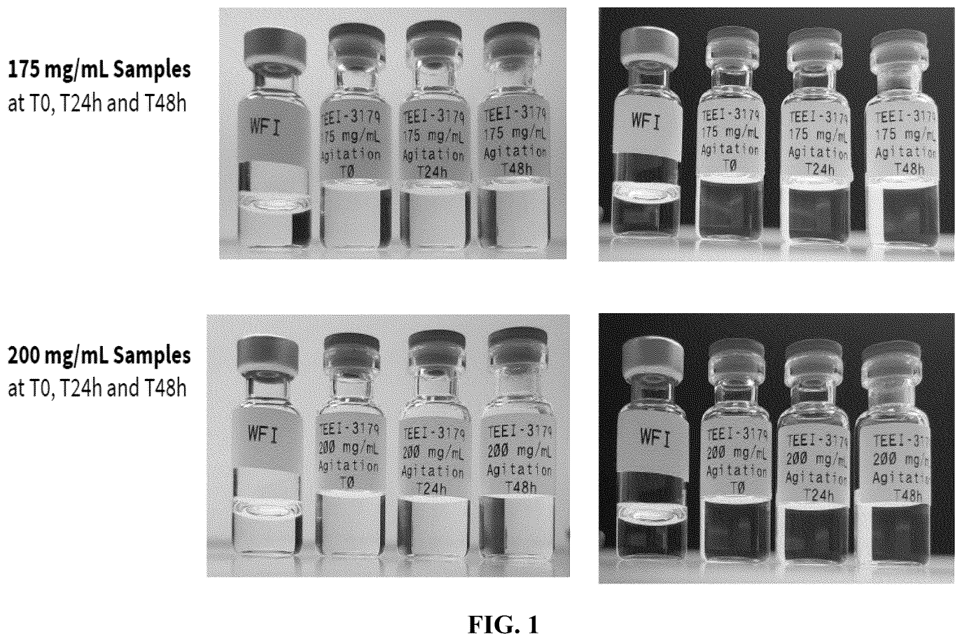

FIG. 1 provides the pictures of samples after agitation study. The material was aliquoted (1.5 mL) into dedicated vials and agitated at 500 rpm (at RT) for a total of 48 h. Samples were analyzed at TO, T24 h and T48 h. The formulations used in this study comprised 25 mM citrate buffer, pH 6, 100 mM arginine, and 0.02% w/v polysorbate 80 as the surfactant. The tested samples had 175 mg/mL and 200 mg/mL budoprutug.

The table below provides a summary of the results from the agitation study.

| Concentration | Timepoint | Visual Appearance |
|---|---|---|
| 175 mg/mL | T0 | V. slight opalescence, v. pale-yellow colour, |
| | | no visible particles |
| | T24 h | V. slight opalescence, v. pale-yellow colour, |
| | | no visible particles |
| | T48 h | V. slight opalescence, v. pale-yellow colour, |
| | | no visible particles |
| 200 mg/mL | T0 | V. slight opalescence, v. pale-yellow colour, |
| | | no visible particles |
| | T24 h | V. slight opalescence, v. pale-yellow colour, |
| | | no visible particles |
| | T48 h | V. slight opalescence, v. pale-yellow colour, |
| | | no visible particles |

The table below provides the results from protein content analysis based on A280 absorption.

| Concentration | Timepoint | Concentration (mg/mL) |
|---|---|---|
| 175 mg/mL | T0 | 173.27 |
| | T24 h | 174.60 |
| | T48 h | 174.74 |
| 200 mg/mL | T0 | 201.72 |
| | T24 h | 205.29 |
| | T48 h | 206.25 |

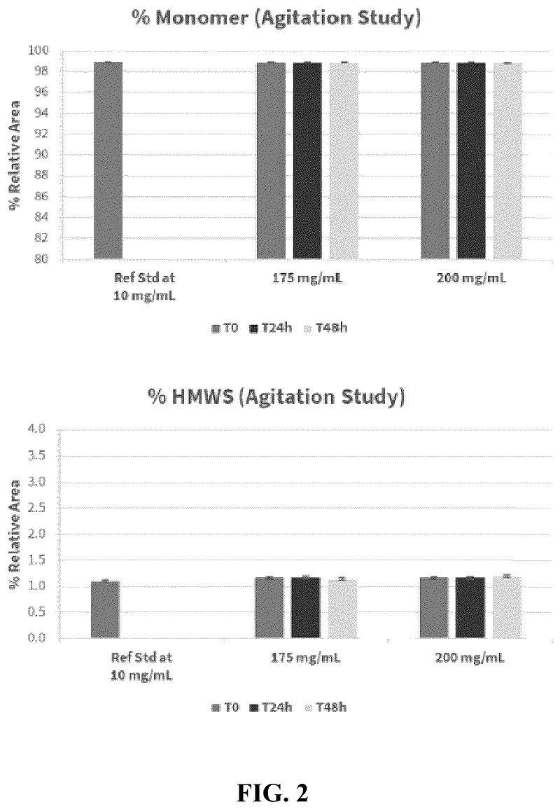

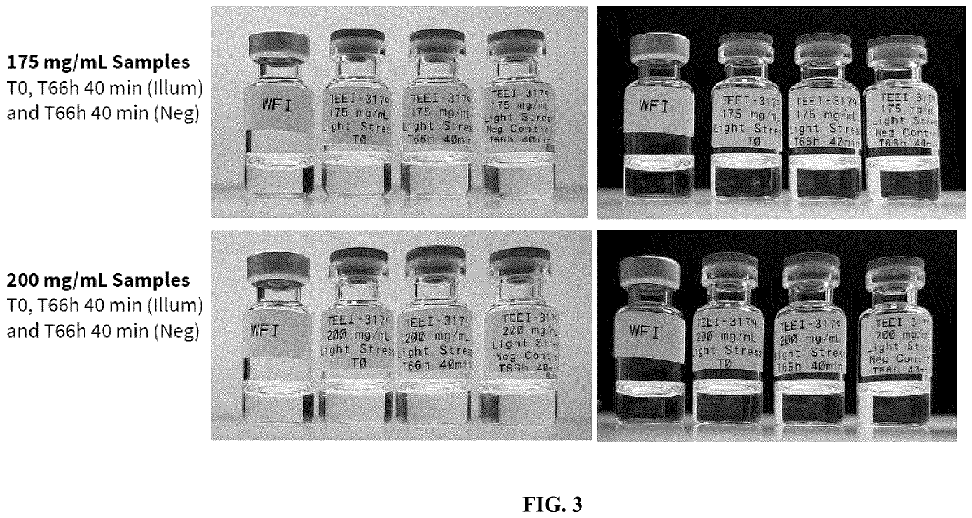

After 48 h of agitation, there were no changes to the % monomer for both concentrations, with levels of HMWS and LMWS remaining the same as TO. FIG. 2 provides the results from the HPLC analysis.

After 48 h, sub-visible particle counts remained low, with very few particles at sizes 10 μm and above. The numbers of particles measured for both concentrations were equivalent to background levels, indicating no increase resulting from agitation stress. The table below provides the results of the particle count analysis.

| | | | All: Particle | All: Conc. |
|---|---|---|---|---|
| Sample | Timepoint | All: % All* | Count (#) | (#/mL) |
| 175 mg/mL | T0 | 96.67 | 29 | 82.67 |
| | T24 h | 85.00 | 34 | 96.65 |
| | T48 h | 95.33 | 102 | 290.77 |
| 200 mg/mL | T0 | 80.85 | 76 | 216.04 |
| | T24 h | 93.33 | 28 | 79.60 |
| | T48 h | 100 | 19 | 54.16 |

### Light Stability Study:

The compositions comprises 175 mg/mL and 200 mg/mL budoprutug. The formulations used in this study comprised 25 mM citrate buffer, pH 6, 100 mM arginine, and 0.02% w/v polysorbate 80 as the surfactant. The tested samples had 175 mg/mL and 200 mg/mL budoprutug.

1.0 mL of each concentration was aliquoted into dedicated 2 mL Type 1 glass vials, with negative control samples wrapped in foil to protect from illumination. Samples were placed in the light chamber and incubated at 25° C./75% RH under cold white-light, for a total of 66 h and 40 min (to reach 1.2 m lux hours at 25° C.).

The table below provides the results from light stability study. Irradiated samples at 175 and 200 mg/mL showed no change in visual appearance compared to the negative control samples, with no change in opalescence, color or formation of visible particles after 66 h 40 min.

| Concentration | Timepoint | Visual Appearance |
|---|---|---|
| 175 mg/mL | T0 | V. slight opalescence, v. pale-yellow |
| | | colour, no visible particles |
| | T66 h 40 | V. slight opalescence, v. pale-yellow |
| | min (Neg) | colour, no visible particles |
| | T66 h 40 | V. slight opalescence, v. pale-yellow |
| | min (Illum) | colour, no visible particles |
| 200 mg/mL | T0 | V. slight opalescence, v. pale-yellow |
| | | colour, no visible particles |
| | T66 h 40 | V. slight opalescence, v. pale-yellow |
| | min (Neg) | colour, no visible particles |
| | T66 h 40 | V. slight opalescence, v. pale-yellow |
| | min (Illum) | colour, no visible particles |

The table below provides the results from protein content analysis based on A280 absorption. No significant changes in protein content were observed for either concentration, after irradiation for 66 h 40 min.

| Concentration | Timepoint | Concentration (mg/mL) |
|---|---|---|
| 175 mg/mL | T0 | 173.27 |
| | T66 h 40 min (Neg) | 175.37 |
| | T66 h 40 min (Illum) | 175.14 |
| 200 mg/mL | T0 | 201.72 |
| | T66 h 40 min (Neg) | 204.08 |
| | T66 h 40 min (Illum) | 204.87 |

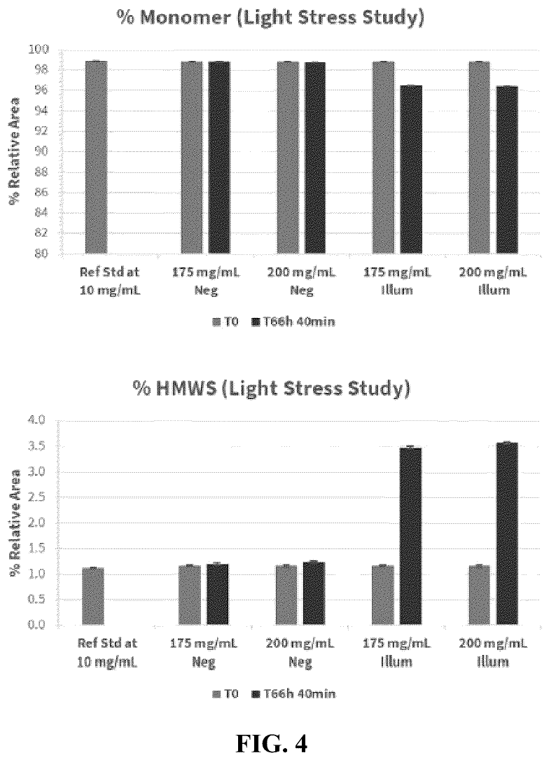

After 66 h 40 min irradiation at 25° C./75% RH, there were notable changes to the irradiated samples at 175 or 200 mg/mL compared to the negative controls, with a ~2.4% increase in HMWS. This could be directly attributable to the exposure to light and not the temperature or humidity. FIG. 4 provides the results from the HPLC analysis.

The table below provides the results from the icIEF analysis. As evident from the table below, After 66 h 40 min irradiation at 25° C./75% RH, there were no significant changes to the charge variant profile for either concentration

| | | % Acidic | % Basic | % Main |
|---|---|---|---|---|
| Concentration | Sample | Species | Species | Species |
| N/A | Ref Std | 19.63 | 8.26 | 72.11 |
| 175 mg/mL | T0 | 19.58 | 8.17 | 72.25 |
| | T66 h 40 min (Neg) | 19.49 | 8.38 | 72.13 |
| | T66 h 40 min (Illum) | 19.84 | 8.32 | 71.84 |
| 200 mg/mL | T0 | 19.27 | 8.31 | 72.41 |
| | T66 h 40 min (Neg) | 19.33 | 8.78 | 71.89 |
| | T66 h 40 min (Illum) | 19.52 | 8.67 | 71.81 |

### Concentration Feasibility Studies:

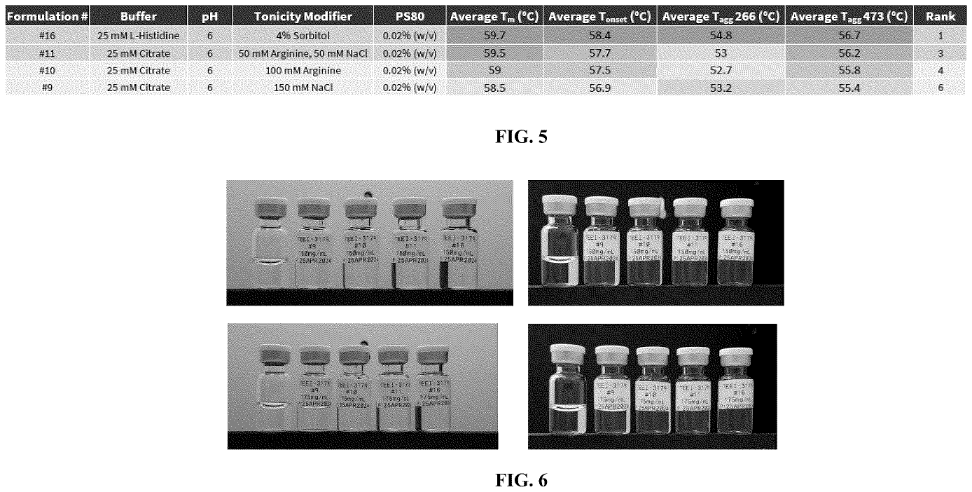

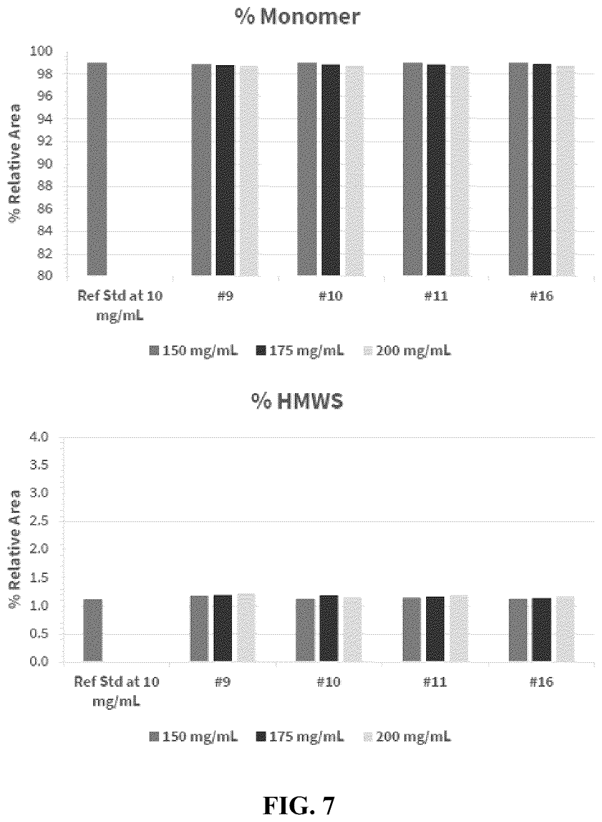

The studies were undertaken to take a high concentration screen. Formulations were developed with several buffers, tonicity modifiers, and surfactants. FIG. 5 provides exemplary formulations comprising budoprutug at varying concentrations. The data provided herein is for exemplary formulations at varying concentrations of budoprutug.

FIG. 6 provides visual appearance results from the concentration feasibility study. The results are summarized in the table below.

| Concen- | Formula- | |
|---|---|---|
| tration | tion # | Visual Appearance |
| 150 mg/mL | #9 | Colourless, very slight opalescence, no visible |
| | | particles |
| | #10 | Colourless, very slight opalescence, no visible |
| | | particles |
| | #11 | Colourless, very slight opalescence, no visible |
| | | particles |
| | #16 | Colourless, very slight opalescence, no visible |
| | | particles |
| 175 mg/mL | #9 | Very slight pale-yellow colouration, very slight |
| | | opalescence, no visible particles |
| | #10 | Very slight pale-yellow colouration, very slight |
| | | opalescence, no visible particles |
| | #11 | Very slight pale-yellow colouration, very slight |
| | | opalescence, no visible particles |
| | #16 | Very slight pale-yellow colouration, very slight |
| | | opalescence, no visible particles |
| 200 mg/mL | #9 | Very slight pale-yellow colouration, very slight |
| | | opalescence, no visible particles |
| | #10 | Very slight pale-yellow colouration, very slight |
| | | opalescence, no visible particles |
| | #11 | Very slight pale-yellow colouration, very slight |
| | | opalescence, no visible particles |
| | #16 | Very slight pale-yellow colouration, very slight |
| | | opalescence, no visible particles |

The Table below provides the results from protein content analysis based on A280 absorption. The samples were diluted 10× to enable measurements to be performed.

| Concentration | Formulation # | Average Value (mg/mL) |
|---|---|---|
| 150 mg/mL | #9 | 146.6 |
| | #10 | 149.3 |
| | #11 | 153.3 |
| | #16 | 153.6 |
| 175 mg/mL | #9 | 171.9 |
| | #10 | 170.8 |
| | #11 | 172.1 |
| | #16 | 173.9 |
| 200 mg/mL | #9 | 201.0 |
| | #10 | 198.1 |
| | #11 | 199.5 |
| | #16 | 196.8 |

The table below provides results of the pH measurements.

Average Value (mOsm/kg)

| Formulation # | 150 mg/mL | 175 mg/mL | 200 mg/mL |
|---|---|---|---|
| #9 | 6.062 | 6.048 | 6.053 |
| #10 | 6.048 | 6.060 | 6.055 |
| #11 | 6.055 | 6.060 | 6.060 |
| #16 | 6.081 | 6.087 | 6.113 |

The table below provides the results for the osmolality measurements for the samples prepared for the concentration feasibility study.

| | Average Value (mOsm/kg) | | | |
|---|---|---|---|---|
| Formulation # | Buffer Only | 150 mg/mL | 175 mg/mL | 200 mg/mL |
| #9 | 401 | 460 | 464 | 488 |
| #10 | 308 | 393 | 399 | 402 |
| #11 | 333 | 398 | 400 | 413 |
| #16 | 347 | 417 | 434 | 440 |

The viscosity measurements at 25° C. are provided in the table below.

Viscosity at 25° C. (cP)

| Formulation # | 150 mg/mL | 175 mg/mL | 200 mg/mL |
|---|---|---|---|
| #9 | 8.29 | 23.10 | 26.19 |
| #10 | 7.47 | 12.18 | 20.81 |
| #11 | 8.17 | 14.38 | 28.56 |
| #16 | 15.14 | 26.35 | 42.87 |

As evident from the table below, all formulations showed acceptable viscosities at 150 mg/mL concentration of the formulation. Indeed, at 175 mg/mL, only formulations 10 and 11 (both comprising viscosity-lowering excipient concentrations) possessed viscosities <20 cP. It is desirable for the viscosity of the formulations to be less than 20 cP so that the compositions may be administered through a disposable syringe. As evident from the table above, the compositions comprising 200 mg/mL, all formulations greater than 20 cP, although some formulations were very close to the limit of the acceptable viscosity.

As evident from the data provided above, the trends observed at 25° C. were also noticed at 4° C. However, the measured viscosity at 4° C., were much higher. These data may have eluded to the fact that only 150 mg/mL, only formulation 10 would likely to injectable formulations. The other formulations at 150 mg/mL and all samples at 175 and 200 mg/mL possessed viscosity values >20 cP, suggesting injection through a disposable syringe would not be possible, and that autoinjectors should be considered if it is not feasible to allow the composition to warm to ambient temperature before injection.

As a result, viscosity reduction studies were undertaken. Subsequently, additional formulations were prepared to test the viscosity of the compositions comprising 200 mg/mL budoprutug. The tested compositions are listed in the table below.

| Formulation # | Buffer | pH | Tonicity Modifier | PS80 |
|---|---|---|---|---|
| #10 | 25 mM | 6 | 100 mM Arginine | 0.02% w/v |
| | Citrate | | | |
| #17 | 25 mM | 6 | 100 mM Arginine | 1% w/v |
| | Citrate | | | |
| #18 | 25 mM | 6 | 100 mM Lysine | 0.02% w/v |
| | Citrate | | | |
| #19 | 25 mM | 6 | 50 mM Arginine, | 0.02% w/v |
| | Citrate | | 50 mM Lysine | |

The table below provides the viscosity for the formulations.

| Concentration | Formulation # | Average Value (cP) |
|---|---|---|
| 200 mg/mL | #10 | 18.42 |
| | #17 | 20.57 |
| | #18 | 24.02 |
| | #19 | 19.08 |

### Thermal Stability Studies:

In conducting the thermal stability studies, the stability study was conducted after stressing the samples under 3 conditions: 2-8° C., 25° C., and 40° C. The measurements were conducted at T0 and T8w at 40° C., T12w at 25° C.; and T26w at 2-8° C. The HPLC analysis for the analysis of samples at T0 is provided below.

| | | | | | Total Peak |
|---|---|---|---|---|---|
| Concen- | | % | % | % | Area |
| tration | Sample | HMWS | LMWS | Monomer | (mAU*min) |
| N/A | DS Ref Std | 1.04 | 0.02 | 98.95 | 45.93 |
| 175 mg/mL | T0 | 1.12 | 0.02 | 98.87 | 44.04 |
| 200 mg/mL | T0 | 1.12 | 0.02 | 98.86 | 45.53 |

These samples displayed similar profiles to the drug substance with no marked increase in aggregation. The measurements after the thermal stability studies also indicated that the samples were stable and had minimal degradation.

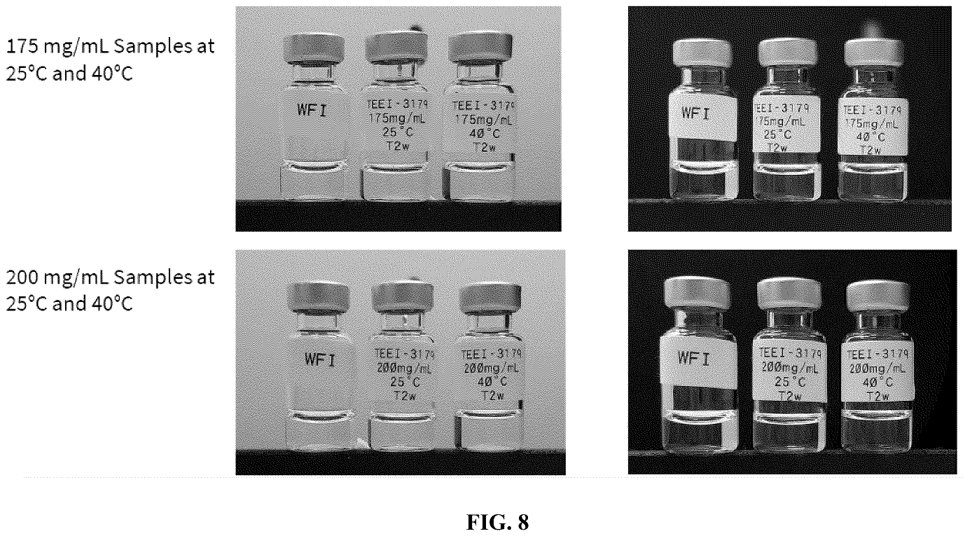

FIG. 8 provides the pictures for samples after the thermal stability studies for 25° C., and 40° C. at 2 weeks. The table below provides the visual appearance studies of these samples:

| | Temper- | Concen- | |
|---|---|---|---|
| Timepoint | ature | tration | Visual Appearance |
| T0 | N/A | 175 mg/mL | V. slight opalescence, v. pale- |
| | | | yellow colour, no visible particles |
| | | 200 mg/mL | V. slight opalescence, v. pale- |
| | | | yellow colour, no visible particles |
| T2 w | 25° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| | 40° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |

The table below provides the results of protein content analysis based on A280 absorption.

| Timepoint | Temperature | Concentration | Average Value (mg/mL) |
|---|---|---|---|
| T0 | N/A | 175 mg/mL | 175.24 |
| | | 200 mg/mL | 201.52 |
| T2 w | 25° C. | 175 mg/mL | 172.65 |
| | | 200 mg/mL | 201.22 |
| | 40° C. | 175 mg/mL | 175.34 |
| | | 200 mg/mL | 201.13 |

The summary of the thermal stability analysis provided in the table below (non-reduced).

| | Temper- | | % Rel. | % Rel. | % |
|---|---|---|---|---|---|
| Timepoint | ature | Sample | Area LC | Area HC | Monomer |
| N/A | N/A | DS Ref Std | 0.12 | n.a. | 98.03 |
| T0 | N/A | 175 mg/mL | 0.12 | n.a. | 97.95 |
| | | 200 mg/mL | 0.20 | n.a. | 97.49 |
| N/A | N/A | DS Ref Std | 0.09 | n.a. | 98.26 |
| T2 w | 25° C. | 175 mg/mL | 0.11 | n.a. | 98.05 |
| | | 200 mg/mL | 0.14 | n.a. | 97.91 |
| | 40° C. | 175 mg/mL | 0.17 | 0.16 | 96.84 |
| | | 200 mg/mL | 0.18 | 0.16 | 96.74 |

After 2 weeks at 25° C., samples at 175 and 200 mg/mL displayed similar levels of monomeric purity compared to T0. After 2 weeks at 40° C., the monomeric purity has decreased slightly for both concentrations, with a ~1% increase in amount of fragmentation. After 2 weeks, the monomeric purity has decreased slightly for both concentrations, with a ~1% increase in amount of fragmentation.

The summary of the thermal stability analysis provided in the table below (non-reduced).

| Time- | Temper- | | % Rel. | % Rel. | Total (% |
|---|---|---|---|---|---|
| point | ature | Sample | Area LC | Area HC | LC + HC) |
| N/A | N/A | DS Ref Std | 25.77 | 72.69 | 98.46 |
| T0 | N/A | 175 mg/mL | 27.06 | 71.42 | 98.48 |
| | | 200 mg/mL | 26.91 | 71.50 | 98.40 |
| N/A | N/A | DS Ref Std | 26.21 | 72.28 | 98.49 |
| T2 w | 25° C. | 175 mg/mL | 26.82 | 70.39 | 97.20 |
| | | 200 mg/mL | 26.74 | 70.47 | 97.21 |
| | 40° C. | 175 mg/mL | 26.81 | 68.00 | 94.81 |
| | | 200 mg/mL | 26.84 | 68.02 | 94.86 |

After 2 weeks at 25° C., samples at 175 and 200 mg/mL displayed slightly decreased summed totals of LC+HC. After 2 weeks at 40° C., samples at 175 and 200 mg/mL displayed a decrease in summed totals of LC+HC of ~3.5%.

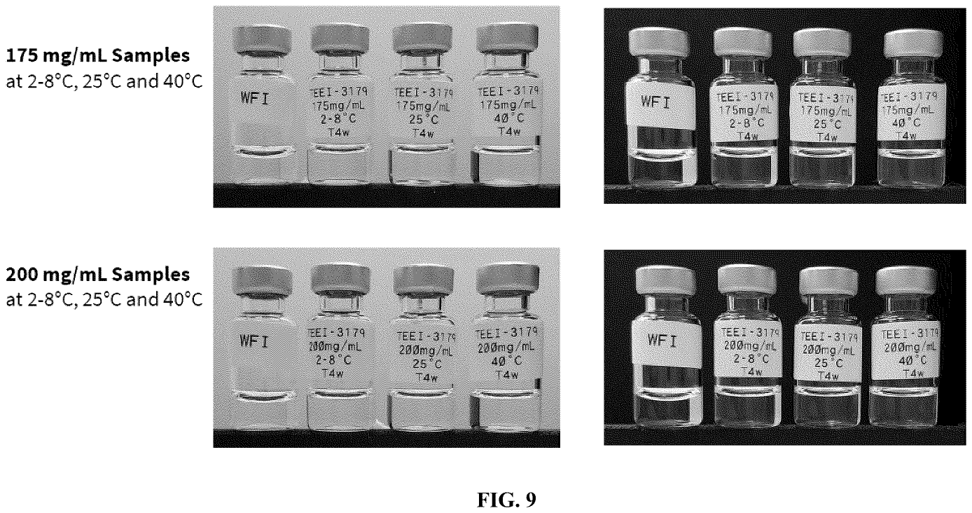

FIG. 9 provides the results from visual appearance from the T4 weeks study for samples stored at 2-8° C., 25° C., and 40° C.

The table below provides the results of the visual analysis from the T4 weeks study for samples stored at 2-8° C., 25° C., and 40° C.

| | Temper- | Concen- | |
|---|---|---|---|
| Timepoint | ature | tration | Visual Appearance |
| T0 | N/A | 175 mg/mL | V. slight opalescence, v. pale- |
| | | | yellow colour, no visible particles |
| | | 200 mg/mL | V. slight opalescence, v. pale- |
| | | | yellow colour, no visible particles |
| T2 w | 25° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| | 40° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| T4 w | 2-8° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| | 25° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| | 40° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |

The table below provides the results of protein content analysis based on A280 absorption.

Average Concentration (mg/mL)

| Temperature | Concentration | T0 | T2 w | T4 w |
|---|---|---|---|---|
| 2-8° C. | 175 mg/mL | 175.24 | N/A | 174.05 |
| | 200 mg/mL | 201.52 | N/A | 204.47 |
| 25° C. | 175 mg/mL | 175.24 | 172.65 | 172.62 |
| | 200 mg/mL | 201.52 | 201.22 | 204.18 |
| 40° C. | 175 mg/mL | 175.24 | 175.34 | 176.24 |
| | 200 mg/mL | 201.52 | 201.13 | 201.82 |

The summary of the thermal stability analysis provided for T4 weeks in the table below at 2-8° C. (non-reduced).

| | Temper- | | % Rel. | % Rel. | % |
|---|---|---|---|---|---|
| Timepoint | ature | Sample | Area LC | Area HC | Monomer |
| N/A | N/A | DS Ref Std | 0.12 | n.a. | 98.03 |
| T0 | N/A | 175 mg/mL | 0.12 | n.a. | 97.95 |
| | | 200 mg/mL | 0.20 | n.a. | 97.49 |
| N/A | N/A | DS Ref Std | 0.79 | n.a. | 90.41 |
| T4 w | 2-8° C. | 175 mg/mL | 0.81 | n.a. | 89.48 |
| | | 200 mg/mL | 0.83 | n.a. | 89.06 |

After 4 weeks at 2-8° C., samples at 175 and 200 mg/mL displayed a slight drop in monomeric purity but was comparable to the DS reference material so could be attributable to variability within the method.

The summary of the thermal stability analysis provided for T4 weeks in the table below at 25° C. (non-reduced).

| | Temper- | | % Rel. | % Rel. | % |
|---|---|---|---|---|---|
| Timepoint | ature | Sample | Area LC | Area HC | Monomer |
| N/A | N/A | DS Ref Std | 0.12 | n.a. | 98.03 |
| T0 | N/A | 175 mg/mL | 0.12 | n.a. | 97.95 |
| | | 200 mg/mL | 0.20 | n.a. | 97.49 |
| N/A | N/A | DS Ref Std | 0.09 | n.a. | 98.26 |
| T2 w | 25° C. | 175 mg/mL | 0.11 | n.a. | 98.05 |
| | | 200 mg/mL | 0.14 | n.a. | 97.91 |
| N/A | N/A | DS Ref Std | 0.79 | n.a. | 90.41 |
| T4 w | 25° C. | 175 mg/mL | 0.81 | n.a. | 87.39 |
| | | 200 mg/mL | 0.75 | n.a. | 87.87 |

After 4 weeks at 25° C., samples at 175 and 200 mg/mL displayed a small drop in monomeric purity (~3%) compared to the product composition reference material.

The summary of the thermal stability analysis provided for T4 weeks in the table below at 40° C. (non-reduced).

| | Temper- | | % Rel. | % Rel. | % |
|---|---|---|---|---|---|
| Timepoint | ature | Sample | Area LC | Area HC | Monomer |
| N/A | N/A | DS Ref Std | 0.12 | n.a. | 98.03 |
| T0 | N/A | 175 mg/mL | 0.12 | n.a. | 97.95 |
| | | 200 mg/mL | 0.20 | n.a. | 97.49 |
| N/A | N/A | DS Ref Std | 0.09 | n.a. | 98.26 |
| T2 w | 40° C. | 175 mg/mL | 0.17 | n.a. | 96.84 |
| | | 200 mg/mL | 0.18 | n.a. | 96.74 |
| N/A | N/A | DS Ref Std | 0.79 | n.a. | 90.41 |
| T4 w | 40° C. | 175 mg/mL | 0.56 | n.a. | 80.31 |
| | | 200 mg/mL | 0.87 | n.a. | 78.08 |

After 4 weeks at 40° C., the monomeric purity has decreased for both concentrations, with a ~10% decrease relative to the DS reference material.

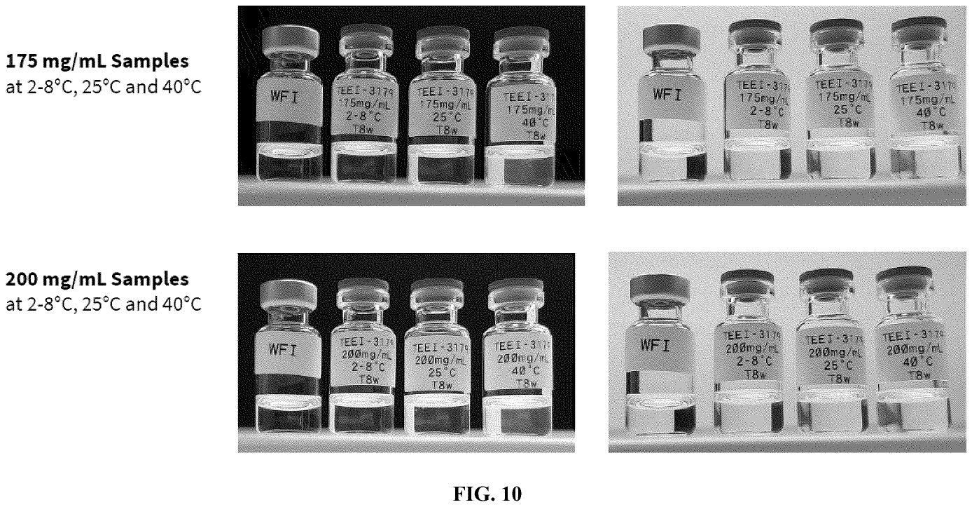

FIG. 10 provides the results from visual appearance from the T8 weeks study for samples stored at 2-8° C., 25° C., and 40° C.

The table below provides the results of the visual analysis from the T8 weeks study for samples stored at 2-8° C., 25° C., and 40° C.

| | Temper- | Concen- | |
|---|---|---|---|
| Timepoint | ature | tration | Visual Appearance |
| T0 | N/A | 175 mg/mL | V. slight opalescence, v. pale- |
| | | | yellow colour, no visible particles |
| | | 200 mg/mL | V. slight opalescence, v. pale- |
| | | | yellow colour, no visible particles |
| T2 w | 25° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| | 40° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| T4 w | 2-8° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| | 25° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| | 40° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| T8 w | 2-8° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| | 25° C. | 175 mg/mL | V. slight opalescence, v. pale- |
| | | | yellow colour, no visible particles |
| | | 200 mg/mL | V. slight opalescence, v. pale- |
| | | | yellow colour, no visible particles |
| | 40° C. | 175 mg/mL | Opalescent, v. pale-yellow colour, |
| | | | no visible particles |
| | | 200 mg/mL | Opalescent, v. pale-yellow colour, |
| | | | no visible particles |

The table below provides the results of protein content analysis based on A280 absorption.

| | Average Concentration (mg/mL) | | | | |
|---|---|---|---|---|---|
| Temperature | Concentration | T0 | T2 w | T4 w | T8 w |
| 2-8° C. | 175 mg/mL | 175.24 | N/A | 174.05 | 177.17 |
| | 200 mg/mL | 201.52 | N/A | 204.47 | 199.09 |
| 25° C. | 175 mg/mL | 175.24 | 172.65 | 172.62 | 173.86 |
| | 200 mg/mL | 201.52 | 201.22 | 204.18 | 198.06 |
| 40° C. | 175 mg/mL | 175.24 | 175.34 | 176.24 | 173.73 |
| | 200 mg/mL | 201.52 | 201.13 | 201.82 | 198.99 |

The summary of the thermal stability analysis provided for T4 weeks in the table below at 2-8 C (non-reduced).

| | | | | | | Total Peak Area |
|---|---|---|---|---|---|---|
| Temperature | Timepoint | Sample | % HMWS | % LMWS | % Monomer | (mAU*min) |
| N/A | N/A | DS Ref Std | 1.04 | 0.02 | 98.95 | 45.93 |
| N/A | T0 | 175 mg/mL | 1.12 | 0.02 | 98.87 | 44.04 |
| | | 200 mg/mL | 1.12 | 0.02 | 98.86 | 45.53 |
| N/A | N/A | DS Ref Std | 1.10 | 0.03 | 98.87 | 49.00 |
| 2-8° C. | T4 w | 175 mg/mL | 1.20 | 0.02 | 98.78 | 44.27 |
| | | 200 mg/mL | 1.22 | 0.03 | 98.75 | 44.29 |
| N/A | N/A | DS Ref Std | 0.99 | 0.03 | 98.98 | 48.92 |
| 2-8° C. | T8 w | 175 mg/mL | 1.15 | 0.02 | 98.84 | 45.90 |
| | | 200 mg/mL | 1.17 | 0.02 | 98.81 | 43.90 |

After 4 weeks at 2-8° C., samples at 175 and 200 mg/mL displayed a slight drop in monomeric purity but was comparable to the product composition reference material so could be attributable to variability within the method.

The HPLC analysis for samples at T4W, and T8W at 2-8° C. are provided below.

| | | | | | | Total Peak Area |
|---|---|---|---|---|---|---|
| Temperature | Timepoint | Sample | % HMWS | % LMWS | % Monomer | (mAU*min) |
| N/A | N/A | DS Ref Std | 1.04 | 0.02 | 98.95 | 45.93 |
| N/A | T0 | 175 mg/mL | 1.12 | 0.02 | 98.87 | 44.04 |
| | | 200 mg/mL | 1.12 | 0.02 | 98.86 | 45.53 |
| N/A | N/A | DS Ref Std | 1.10 | 0.03 | 98.87 | 49.00 |
| 2-8° C. | T4 w | 175 mg/mL | 1.20 | 0.02 | 98.78 | 44.27 |
| | | 200 mg/mL | 1.22 | 0.03 | 98.75 | 44.29 |
| N/A | N/A | DS Ref Std | 0.99 | 0.03 | 98.98 | 48.92 |
| 2-8° C. | T8 w | 175 mg/mL | 1.15 | 0.02 | 98.84 | 45.90 |
| | | 200 mg/mL | 1.17 | 0.02 | 98.81 | 43.90 |

After 8 weeks at 2-8° C., samples at 175 and 200 mg/mL displayed no change to the monomeric content compared to T0.

The HPLC analysis for samples at T4W, and T8W at 25° C. are provided below.

| | | | | | | Total Peak Area |
|---|---|---|---|---|---|---|
| Temperature | Timepoint | Sample | % HMWS | % LMWS | % Monomer | (mAU*min) |
| N/A | N/A | DS Ref Std | 1.04 | 0.02 | 98.95 | 45.93 |
| N/A | T0 | 175 mg/mL | 1.12 | 0.02 | 98.87 | 44.04 |
| | | 200 mg/mL | 1.12 | 0.02 | 98.86 | 45.53 |
| N/A | N/A | DS Ref Std | 1.08 | 0.03 | 98.90 | 46.51 |
| 25° C. | T2 w | 175 mg/mL | 1.22 | 0.04 | 98.74 | 43.75 |
| | | 200 mg/mL | 1.24 | 0.04 | 98.73 | 43.66 |
| N/A | N/A | DS Ref Std | 1.10 | 0.03 | 98.87 | 49.00 |
| 25° C. | T4 w | 175 mg/mL | 1.37 | 0.46 | 98.17 | 46.56 |
| | | 200 mg/mL | 1.40 | 0.47 | 98.14 | 46.34 |
| N/A | N/A | DS Ref Std | 0.99 | 0.03 | 98.98 | 48.92 |
| 25° C. | T8 w | 175 mg/mL | 1.35 | 0.76 | 97.90 | 44.73 |
| | | 200 mg/mL | 1.39 | 0.79 | 97.83 | 42.92 |

After 8 weeks at 25° C., samples at 175 and 200 mg/mL displayed a small decrease in the monomeric content, with a small increase in % HMWS compared to T0. Levels of LMWS increased compared to T4w, indicating occurrence of fragmentation.

The HPLC analysis for samples at T4W, and T8W at 40° C. are provided below.

| | | | | | | Total Peak Area |
|---|---|---|---|---|---|---|
| Temperature | Timepoint | Sample | % HMWS | % LMWS | % Monomer | (mAU*min) |
| N/A | N/A | DS Ref Std | 1.04 | 0.02 | 98.95 | 45.93 |
| N/A | T0 | 175 mg/mL | 1.12 | 0.02 | 98.87 | 44.04 |
| | | 200 mg/mL | 1.12 | 0.02 | 98.86 | 45.53 |
| N/A | N/A | DS Ref Std | 1.08 | 0.03 | 98.90 | 46.51 |
| 40° C. | T2 w | 175 mg/mL | 1.91 | 1.13 | 96.96 | 42.35 |
| | | 200 mg/mL | 1.88 | 1.14 | 96.99 | 43.62 |
| N/A | N/A | DS Ref Std | 1.10 | 0.03 | 98.87 | 49.00 |
| 40° C. | T4 w | 175 mg/mL | 3.02 | 2.23 | 94.76 | 43.80 |
| | | 200 mg/mL | 2.80 | 2.09 | 95.12 | 43.43 |
| N/A | N/A | DS Ref Std | 0.99 | 0.03 | 98.98 | 48.92 |
| 40° C. | T8 w | 175 mg/mL | 5.00 | 4.39 | 90.62 | 44.28 |
| | | 200 mg/mL | 4.48 | 3.90 | 91.63 | 42.87 |

After 8 weeks at 40° C., samples at 175 and 200 mg/mL displayed a ~8% drop in the monomeric content due to increases in both % HMWS and LMWS.

The summary of the thermal stability analysis provided for T8 weeks in the table below at 2-8° C. (reduced).

| Time- | Temper- | | % Rel. | % Rel. | Total (% |
|---|---|---|---|---|---|
| point | ature | Sample | Area LC | Area HC | LC + HC) |
| N/A | N/A | DS Ref Std | 25.77 | 72.69 | 98.46 |
| T0 | N/A | 175 mg/mL | 27.06 | 71.42 | 98.48 |
| | | 200 mg/mL | 26.91 | 71.50 | 98.40 |
| N/A | N/A | DS Ref Std | 25.43 | 73.04 | 98.48 |
| T4 w | 2-8° C. | 175 mg/mL | 26.69 | 71.51 | 98.20 |
| | | 200 mg/mL | 26.27 | 72.44 | 98.71 |
| N/A | N/A | DS Ref Std | 25.50 | 72.83 | 98.33 |
| T8 w | 2-8° C. | 175 mg/mL | 26.77 | 71.24 | 98.02 |
| | | 200 mg/mL | 26.84 | 71.12 | 97.96 |

After 8 weeks at 2-8° C., the summed totals of LC+HC for samples at 175 and 200 mg/mL were relatively unchanged compared to T0 and T4w.

The summary of the thermal stability analysis provided for T8 weeks in the table below at 25° C. (reduced).

| Time- | Temper- | | % Rel. | % Rel. | Total (% |
|---|---|---|---|---|---|
| point | ature | Sample | Area LC | Area HC | LC + HC) |
| N/A | N/A | DS Ref Std | 25.77 | 72.69 | 98.46 |
| T0 | N/A | 175 mg/mL | 27.06 | 71.42 | 98.48 |
| | | 200 mg/mL | 26.91 | 71.50 | 98.40 |
| N/A | N/A | DS Ref Std | 26.21 | 72.28 | 98.49 |
| T2 w | 25° C. | 175 mg/mL | 26.82 | 70.39 | 97.20 |
| | | 200 mg/mL | 26.74 | 70.47 | 97.21 |
| N/A | N/A | DS Ref Std | 25.43 | 73.04 | 98.48 |
| T4 w | 25° C. | 175 mg/mL | 26.60 | 70.78 | 97.38 |
| | | 200 mg/mL | 26.75 | 70.61 | 97.37 |
| N/A | N/A | DS Ref Std | 25.50 | 72.83 | 98.33 |
| T8 w | 25° C. | 175 mg/mL | 26.64 | 69.26 | 95.90 |
| | | 200 mg/mL | 26.60 | 69.26 | 95.86 |

After 8 weeks at 25° C., summed totals of LC+HC for samples at 175 and 200 mg/mL displayed a small decrease of ~2.5%.

As demonstrated in the table below, after 8 weeks at 40° C., samples at 175 and 200 mg/mL displayed a decrease in summed totals of LC+HC of ~12%.

| Time- | Temper- | | % Rel. | % Rel. | Total (% |
|---|---|---|---|---|---|
| point | ature | Sample | Area LC | Area HC | LC + HC) |
| N/A | N/A | DS Ref Std | 25.77 | 72.69 | 98.46 |
| T0 | N/A | 175 mg/mL | 27.06 | 71.42 | 98.48 |
| | | 200 mg/mL | 26.91 | 71.50 | 98.40 |
| N/A | N/A | DS Ref Std | 26.21 | 72.28 | 98.49 |
| T2 w | 40° C. | 175 mg/mL | 26.81 | 68.00 | 94.81 |
| | | 200 mg/mL | 26.84 | 68.02 | 94.86 |
| N/A | N/A | DS Ref Std | 25.43 | 73.04 | 98.48 |
| T4 w | 40° C. | 175 mg/mL | 26.58 | 65.77 | 92.35 |
| | | 200 mg/mL | 26.44 | 66.10 | 92.54 |
| N/A | N/A | DS Ref Std | 25.50 | 72.83 | 98.33 |
| T8 w | 40° C. | 175 mg/mL | 25.54 | 60.98 | 86.52 |
| | | 200 mg/mL | 25.71 | 61.39 | 87.10 |

A summary MFI analysis is provided below. As demonstrated in the table below, after 8 weeks at 40° C., samples at 175 and 200 mg/mL displayed an increase in sub-visible particles, indicating an increase in protein aggregation.

| | | | All: Particle | All: Conc. |
|---|---|---|---|---|
| Sample | Timepoint | All: % All* | Count (#) | (#/mL) |
| 175 mg/mL | T0 | 100 | 33 | 94.07 |
| | T8 w at 40° C. | 96.77 | 599 | 1707.57 |
| 200 mg/mL | T0 | 80 | 16 | 45.61 |
| | T8 w at 40° C. | 96.64 | 1092 | 3112.97 |

The table below provides the sequences for nucleic acids, proteins, and peptides discussed herein.

| SEQ ID | |
|---|---|
| NO: | Sequence |
| 1 | CAGGTGCAACTGCAGCAGCCTGGGGCTGAAGTGGTGAAGCCTGGGGC |
| | TTCAGTGAGACTGTCCTGCAAGACTTCTGGCTACACCTTCACCAGCAA |
| | CTGGATGCACTGGGTGAAGCAGAGGCCTGGACAAGGCCTTGAGTGGA |
| | TCGGAGAGATTGATCCTTCTGATAGTTATACTAACTACAATCAAAAGTT |
| | CAAGGGCAAGGCCAAGTTGACTGTAGACAAATCCTCCAGCACAGCCT |
| | ACATGGAAGTCAGCAGCCTGACATCTGAGGACTCTGCGGTCTATTACT |
| | GTGCAAGAGGTAGCAACCCTTACTACTATGCTATGGACTACTGGGGTCA |
| | AGGAACCTCAGTCACCGTCTCCTCA |
| 2 | CAGGTGCAACTGCAGCAGCCTGGGGCTGAAGTGGTGAAGCCTGGGGC |
| | TTCAGTGAGACTGTCCTGCGAGACTTCTGGCTACACCTTCACCAGCAA |
| | CTGGATGCACTGGGTGAAGCAGAGGCCTGACCAAGGACTTGAGTGGA |
| | TCGGAGAGATTGATCCTTCTGATAGTTATACTAACTACAATCAAAAGTT |
| | CAAGGGCAAGGCCGAATTGACTGTAGACAAATCCTCCAGCACAGCCTA |
| | CATGGAAGTCAGCGACCTGACATCTGAGGACTCTGCGGTCTATTACTGT |
| | GCAAGAGGTAGCAACCCTTACTACTATGCTATGGACTACTGGGGTCAA |
| | GGAACCTCAGTCACCGTCTCCTCA |
| 3 | CAGGTGCAACTGCAGCAGCCTGGGGCTGAAGTGGTGAAGCCTGGGGC |
| | TTCAGTGAGACTGTCCTGCAAGACTTCTGGCTACACCTTCACCAGCAA |
| | CTGGATGCACTGGGTGAAGCAGAGACCTGGACAAGGACTTGAGTGGA |
| | TCGGAGAGATTGATCCTTCTGATAGTTATACTAACTACAATCAAAAGTT |
| | CAAGGGCAAGGCCGAATTGACTGTAGACAAATCCTCCAGCACAGCCTA |
| | CATGGAAGTCAGCGACCTGACATCTGAGGACTCTGCGGTCTATTACTGT |
| | GCAAGAGGTAGCAACCCTTACTACTATGCTATGGACTACTGGGGTCAA |
| | GGAACCTCAGTCACCGTCTCCTCA |
| 4 | CAGGTGCAACTGGAGCAGCCTGGGGCTGAAGTGAAGAAGCCTGGGGC |
| | TTCAGTGAAGGTGTCCTGCAAGGCTTCTGGCTACACCTTCACCAGCAA |
| | CTGGATGCACTGGGTGAAGCAGAGGCCTGGACAAGGACTTGAGTGGA |
| | TCGGAGAGATTGATCCTTCTGATAGTTATACTAACTACAATCAAAAGTT |
| | CAAGGGCAAGGCCAAGTTGACTGTAGACAAATCCTCCAGCACAGCCT |
| | ACATGGAAGTCAGCGACCTGACAGCTGAGGACTCTGCGGTCTATTACT |
| | GTGCAAGAGGTAGCAACCCTTACTACTATGCTATGGACTACTGGGGTCA |
| | AGGAACCTCAGTCACCGTCTCCTCA |
| 5 | CAGGTGCAACTGGAGCAGCCTGGGGCTGAAGTGGTGAAGCCTGGGGC |
| | TTCAGTGAAGGTGTCCTGCAAGACTTCTGGCTACACCTTCACCAGCAA |
| | CTGGATGCACTGGGTGAAGCAGACGCCTGGAAAAGGACTTGAGTGGA |
| | TCGGAGAGATTGATCCTTCTGATAGTTATACTAACTACAATCAAAAGTT |
| | CGATGGCAAGGCCAAGTTGACTGTAGACAAATCCTCCAGCACAGCCTA |
| | CATGGAAGTCAGCGACCTGACAGCTGAGGACTCTGCGACCTATTACTG |
| | TGCAAGAGGTAGCAACCCTTACTACTATGCTATGGACTACTGGGGTCAA |
| | GGAACCTCAGTCACCGTCTCCTCA |
| 6 | CAGGTGCAACTGGAGCAGCCTGGGGCTGAAGTGAAGAAGCCTGGGGC |
| | TTCAGTGAAGGTGTCCTGCAAGGCTTCTGGCTACACCTTCACCAGCAA |
| | CTGGATGCACTGGGTGAGACAGGCACCTGGAAAAGGACTTGAGTGGA |
| | TCGGAGAGATTGATCCTTCTGATAGTTATACTAACTACAATCAAAAGTT |
| | CGATGGCAAGGCCAAGTTGACTGTAGACAAATCCTCCAGCACAGCCTA |
| | CATGGAAGTCAGCGACCTGACATCTGAGGACTCTGCGACCTATTACTG |
| | TGCAAGAGGTAGCAACCCTTACTACTATGCTATGGACTACTGGGGTCAA |
| | GGAACCTCAGTCACCGTCTCCTCA |
| 7 | CAGGTGCAACTGGAGCAGCCTGGGGCTGAAGTGGTGAAGCCTGGGGC |
| | TTCAGTGAAGGTGTCCTGCAAGACTTCTGGCTACACCTTCACCAGCAA |
| | CTGGATGCACTGGGTGAAGCAGAGGCCTGGACAAGGACTTGAGTGGA |
| | TCGGAGAGATTGATCCTTCTGATAGTTATACTAACTACAATCAAAAGTT |
| | CGATGGCAAGGCCAAGTTGACTGTAGACAAATCCTCCAGCACAGCCTA |
| | CATGGAAGTCAGCGACCTGACATCTGAGGACTCTGCGACCTATTACTG |
| | TGCAAGAGGTAGCAACCCTTACTACTATGCTATGGACTACTGGGGTCAA |
| | GGAACCTCAGTCACCGTCTCCTCA |
| 8 | CAAATTGTTCTCACCCAGTCTCCAGCAATCATGTCTGCATCTCCAGGGG |
| | AGAAGGTCACCATGACCTGCAGTGCCAGCTCAGGTGTCAACTACATGC |
| | ACTGGTACCAGCAGAAGCCAGGCACCTCCCCCAAAAGATGGATTTATG |
| | ACACATCCAAACTGGCTTCTGGAGTCCCTGCTCGCTTCAGTGGCAGTG |
| | GGTCTGGGACCTCTTATTCTCTCACAATCAGCAGCATGGAGGCTGAAG |
| | ATGCTGCCACTTATTACTGCCATCAGCGAGGTAGTTACACGTTCGGAGG |
| | GGGGACCAAGCTGGAAATAAA |
| 9 | CAAATTGCTCTCACCCAGGAGCCAGCAATCATGTCTGCATCTCCAGGG |
| | GAGAAGGTCACCATGACCTGCAGTGCCAGCTCAGGTGTCAACTACATG |
| | CACTGGTATCAGCAGAAGCCAGGCACCTCCCCCAAAAGATGGATTTAT |
| | GACACATCCAAACTGGATTCTGGAGTCCCTGCTCGCTTCAGTGGCAGT |
| | GGGTCTGGGACCTCTTATTCTCTCACAATCAGCAGCATGGAGGCTGAA |
| | GATGCTGCCACTTATTACTGCCATCAGCGAGGTAGTTACACGTTCGGAG |
| | GGGGGACCAAGCTGGAAATAAA |
| 10 | GACATTGTTCTCACCCAGTCTCCAGCAACTTTGTCTGCATCTCCAGGGG |
| | AGAAGGTCACCATGACCTGTAGTGCCAGCTCAGGTGTCAACTACATGC |
| | ACTGGTATCAGCAGAAGCCAGGCACCTCCCCCAAAAGATGGATTTATG |
| | ACACATCCAAACTGGATTCTGGAGTCCCTGCTCGCTTCAGTGGCAGTG |
| | GGTCTGGGACCTCTTATTCTCTCACAATCAGCAGCATGGAGGCTGAAG |
| | ATGCTGCCACTTATTACTGCCATCAGCGAGGTAGTTACACGTTCGGAGG |
| | GGGGACCAAGCTGGAAATAAA |
| 11 | CAAATTGTTCTCACCCAGTCTCCAGCAACTTTGTCTGCATCTCCAGGGG |
| | AGAAGGCTACCATGACCTGCAGTGCCAGCTCAGGTGCTAACTACATGC |
| | ACTGGTACCAGCAGAAGCCAGGCACCTCCCCCAAAAGATGGATTTATG |
| | ACACATCCAAACTGGCTTCTGGAGTCCCTGCTCGCTTCAGTGGCAGTG |
| | GGTCTGGGACCTCTTATTCTCTCACAATCGAGAGCATGGAGGCTGAAG |
| | ATGCTGCCACTTATTACTGCCATCAGCGAGGTAGTTACACGTTCGGAGG |
| | GGGGACCAAGCTGGAAATAAA |
| 12 | CAAATTGTTCTCACCCAGTCTCCAGCAACTTTGTCTGCATCTCCAGGGG |
| | AGAAGGCTACATGACCTGTAGTGCCAGCTCAGGTGTCAACTACATGCA |
| | CTGGTACCAGCAGAAGCCAGGCACCTCCCCCAAAAGATGGATTTATGA |
| | CACAGACAAAACGGCTTCTGGAGTCCCTGCTCGCTTCAGTGGCAGTGG |
| | GTCTGGGACCTCTTATTCTCTCACAATCAGCAGCATGGAGGCTGAAGAT |
| | GCTGCCACTTATTACTGCCATCAGCGAGGTAGTTACACGTTCGGAGGG |
| | GGGACCAAGCTGGAAATAAA |
| 13 | QVQLQQPGAEVVKPGASVRLSCKTSGYTFTSNWMHWVKQRPGQGLEWI |
| | GEIDPSDSYTNYNQKFKGKAKLTVDKSSSTAYMEVSSLTSEDSAVYYCAR |
| | GSNPYYYAMDYWGQGTSVTVSS |
| 14 | QVQLQQPGAEVVKPGASVRLSCETSGYTFTSNWMHWVKQRPDQGLEWI |
| | GEIDPSDSYTNYNQKFKGKAELTVDKSSSTAYMEVSDLTSEDSAVYYCAR |
| | GSNPYYYAMDYWGQGTSVTVSS |
| 15 | QVQLQQPGAEVVKPGASVRLSCKTSGYTFTSNWMHWVKQRPGQGLEWI |
| | GEIDPSDSYTNYNQKFKGKAELTVDKSSSTAYMEVSDLTSEDSAVYYCAR |
| | GSNPYYYAMDYWGQGTSVTVSS |
| 16 | QVQLEQPGAEVKKPGASVKVSCKASGYTFTSNWMHWVKQRPGQGLEW |
| | IGEIDPSDSYTNYNQKFKGKAKLTVDKSSSTAYMEVSDLTAEDSAVYYCA |
| | RGSNPYYYAMDYWGQGTSVTVSS |
| 17 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSS |
| 18 | QVQLEQPGAEVKKPGASVKVSCKASGYTFTSNWMHWVRQAPGKGLEW |
| | IGEIDPSDSYTNYNQKFDGKAKLTVDKSSSTAYMEVSDLTSEDSATYYCA |
| | RGSNPYYYAMDYWGQGTSVTVSS |
| 19 | QVQLEQPGAEVVKPGASVKVSCKTSGYTFTSNWMHWVKQRPGQGLEWI |
| | GEIDPSDSYTNYNQKFDGKAKLTVDKSSSTAYMEVSDLTSEDSATYYCAR |
| | GSNPYYYAMDYWGQGTSVTVSS |
| 20 | QVQLQQPGAEVKKPGASVRLSCETSGYTFTSNWMHWVKQRPDQGLEWI |
| | GEIDPSDSYTNYNQKFDGKAELTVDKSSSTAYMEVSDLTSEDSAVYYCAR |
| | GSNPYYYAMDYWGQGTSVTVSS |
| 21 | QVQLEQPGAEVKKPGASVKVSCKASGYTFTSNWMHWVKQTPGKGLEW |
| | IGEIDPSDSYTNYNQKFDGKAKLTVDKSSSTAYMEVSDLTAEDSATYYCA |
| | RGSNPYYYAMDYWGQGTSVTVSS |
| 22 | QVQLXQPGAEVXKPGASVXXSCXXSGYTF |
| 23 | WVXQXPXXGLEWIG |
| 24 | YNQKFXGKAXLTVDKSSSTAYMEVSXLTXEDSAXYYCA |
| 25 | QIVLTQSPAIMSASPGEKVTMTCSASSGVNYMHWYQQKPGTSPKRWIYD |
| | TSKLASGVPARFSGSGSGTSYSLTISSMEAEDAATYYCHQRGSYTFGGGT |
| | KLEIK |
| 26 | QIALTQEPAIMSASPGEKVTMTCSASSGVNYMHWYQQKPGTSPKRWIYD |
| | TSKLDSGVPARFSGSGSGTSYSLTISSMEAEDAATYYCHQRGSYTFGGGT |
| | KLEIK |
| 27 | DIVLTQSPATLSASPGEKVTMTCSASSGVNYMHWYQQKPGTSPKRWIYDT |
| | SKLDSGVPARFSGSGSGTSYSLTISSMEAEDAATYYCHQRGSYTFGGGTK |
| | LEIK |
| 28 | QIVLTQSPATLSASPGEKATMTCSASSGANYMHWYQQKPGTSPKRWIYDT |
| | SKLASGVPARFSGSGSGTSYSLTIESMEAEDAATYYCHQRGSYTFGGGTK |
| | LEIK |
| 29 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIK |
| 30 | QIALTQEPAIMSASPGEKATMTCSASSGVNYMHWYQQKPGTSPKRWIYD |
| | TSKLDSGVPARFSGSGSGTSYSLTIESMEAEDAATYYCHQRGSYTFGGGT |
| | KLEIK |
| 31 | QIVLTQSPATLSASPGEKATMTCSASSGANYMHWYQQKPGTSPKRWIYDT |
| | DKTASGVPARFSGSGSGTSYSLTIESMEAEDAATYYCHQRGSYTFGGGTK |
| | LEIK |
| 32 | XIXLTQXPAXXSASPGEKXTMTC |
| 33 | DTXKXX |
| 34 | QVQLQQPGAEVVKPGASVRLSCKTSGYTFTSNWMHWVKQRPGQGLEWI |
| | GEIDPSDSYTNYNQKFKGKAKLTVDKSSSTAYMEVSSLTSEDSAVYYCAR |
| | GSNPYYYAMDYWGQGTSVTVSS |
| 35 | QIVLTQSPAIMSASPGEKVTMTCSASSGVNYMHWYQQKPGTSPKRWIYD |
| | TSKLASGVPARFSGSGSGTSYSLTISSMEAEDAATYYCHQRGSYTFGGGT |
| | KLEIK |
| 36 | VRLSCKTSG |
| 37 | WVKQRPGQG |
| 38 | YNQKFKGKA |
| 39 | FKGKAKLTV |
| 40 | YMEVSSLTS |
| 41 | VYYCARGSN |
| 42 | IVLTQSPAI |
| 43 | VLTQSPAIM |
| 44 | VTMTCSASS |
| 45 | VNYMHWYQQ |
| 46 | WIYDTSKLA |
| 47 | IYDTSKLAS |
| 48 | CGTAAGTGGATCC |
| 49 | GCTAGCTCCAGC |
| 50 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLS |
| | PGK |
| 51 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLS |
| | PGK |
| 52 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |
| 53 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |
| 54 | QVQLEQPGAEVVKPGASVKVSCKTSGYTFTSNWMHWVKQTPGKGLEWI |
| | GEIDPSDSYTNYNQKFDGKAKLTVDKSSSTAYMEVSDLTAEDSATYYCAR |
| | GSNPYYYAMDYWGQGTSVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLS |
| | PGK |
| 55 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTK |
| | LEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A composition comprising a therapeutically effective amount of an anti-CD19 antibody, wherein the anti-CD19 antibody comprises: a heavy chain variable region at least 95% identical to SEQ ID NO: 17 and/or a light chain variable region at least 95% identical to SEQ ID NO: 29; and wherein the composition comprises at least about 100 mg/ml of the anti-CD19 antibody.

2. The composition of claim 1, wherein the composition further comprises a pharmaceutically acceptable excipient.

3. The composition of claim 1, wherein the composition further comprises a buffer.

4. The composition of claim 1, wherein the composition further comprises a tonicity modifier.

5. The composition of claim 1, wherein the anti-CD19 antibody is budoprutug.

6. The composition of claim 1, wherein the composition comprises a pharmaceutically acceptable buffer, a tonicity modifier, and/or a surfactant.

7. The composition of claim 1, wherein the composition comprises from about 100 mg/mL to about 300 mg/mL of the anti-CD19 antibody.

8. The composition of claim 1, wherein the composition comprises about 100 mg/mL of the anti-CD19 antibody.

9. The composition of claim 1, wherein the composition comprises about 175 mg/mL of the anti-CD19 antibody.

10. The composition of claim 1, wherein the composition comprises about 200 mg/ml of the anti-CD19 antibody.

11. The composition of claim 6, wherein the pharmaceutically acceptable buffer is selected from the group consisting of citrate, acetate, tromethamine, phosphate, hydroxymethylaminoethane, histidine, and glutamate.

12. The composition of claim 11, wherein the composition comprises about 10 mM to about 100 mM of the pharmaceutically acceptable buffer.

13. The composition of claim 11, wherein the composition comprises about 10 mM to about 50 mM of the pharmaceutically acceptable buffer.

14. The composition of claim 11, wherein the composition comprises about 25 mM of the pharmaceutically acceptable buffer.

15. The composition of claim 11, wherein the pharmaceutically acceptable buffer is citrate.

16. The composition of claim 11, wherein the pharmaceutically acceptable buffer is histidine.

17. The composition of claim 6, wherein in the tonicity modifier is selected from the group consisting of sodium chloride, arginine, sorbitol, dextrose, mannitol, potassium chloride, maltose, trehalose, glycerol, or any combination thereof.

18. The composition of claim 17, wherein the composition comprises from about 50 mM to about 200 mM of the tonicity modifier.

19. The composition of claim 17, wherein the composition comprises from about 1% to about 10% w/v of the tonicity modifier.

20. The composition of claim 17, wherein the composition comprises from about 5% w/v of the tonicity modifier.

21. The composition of claim 18, wherein the composition comprises about 100 mM of the tonicity modifier.

22. The composition of claim 18, wherein the composition comprises about 150 mM of the tonicity modifier.

23. The composition of claim 17, wherein the tonicity modifier is selected from the group consisting of sodium chloride, arginine, sorbitol, and any combination thereof.

24. The composition of claim 17, wherein the tonicity modifier is selected from the group consisting of sodium chloride, arginine, and any combination thereof.

25. The composition of claim 6, wherein the surfactant is a polysorbate.

26. The composition of claim 6, wherein the surfactant is polysorbate 80.

27. The composition of claim 6, wherein the pH of the composition is from about 5 to about 7.

28. The composition of claim 6, wherein the pH of the composition is about 6.

29. The composition of claim 3, wherein the viscosity of the composition is less than 30 cP.

30. The composition of any of claim 3, wherein the viscosity of the composition is less than 20 cP.


SEQUENCE LISTING (55 sequences)

SEQ ID NO: 1 - DNA, 360 bp, synthetic construct
  source 1..360: mol_type = other DNA
caggtgcaactgcagcagcctggggctgaagtggtgaagcctggggcttcagtgagactgtcctgcaagacttctggctacaccttcaccagcaactggatgcactgggtgaagcagaggcctggacaaggccttgagtggatcggagagattgatccttctgatagttatactaactacaatcaaaagttcaagggcaaggccaagttgactgtagacaaatcctccagcacagcctacatggaagtcagcagcctgacatctgaggactctgcggtctattactgtgcaagaggtagcaacccttactactatgctatggactactggggtcaaggaacctcagtcaccgtctcctca

SEQ ID NO: 2 - DNA, 360 bp, synthetic construct
  source 1..360: mol_type = other DNA
caggtgcaactgcagcagcctggggctgaagtggtgaagcctggggcttcagtgagactgtcctgcgagacttctggctacaccttcaccagcaactggatgcactgggtgaagcagaggcctgaccaaggacttgagtggatcggagagattgatccttctgatagttatactaactacaatcaaaagttcaagggcaaggccgaattgactgtagacaaatcctccagcacagcctacatggaagtcagcgacctgacatctgaggactctgcggtctattactgtgcaagaggtagcaacccttactactatgctatggactactggggtcaaggaacctcagtcaccgtctcctca

SEQ ID NO: 3 - DNA, 360 bp, synthetic construct
  source 1..360: mol_type = other DNA
caggtgcaactgcagcagcctggggctgaagtggtgaagcctggggcttcagtgagactgtcctgcaagacttctggctacaccttcaccagcaactggatgcactgggtgaagcagagacctggacaaggacttgagtggatcggagagattgatccttctgatagttatactaactacaatcaaaagttcaagggcaaggccgaattgactgtagacaaatcctccagcacagcctacatggaagtcagcgacctgacatctgaggactctgcggtctattactgtgcaagaggtagcaacccttactactatgctatggactactggggtcaaggaacctcagtcaccgtctcctca

SEQ ID NO: 4 - DNA, 360 bp, synthetic construct
  source 1..360: mol_type = other DNA
caggtgcaactggagcagcctggggctgaagtgaagaagcctggggcttcagtgaaggtgtcctgcaaggcttctggctacaccttcaccagcaactggatgcactgggtgaagcagaggcctggacaaggacttgagtggatcggagagattgatccttctgatagttatactaactacaatcaaaagttcaagggcaaggccaagttgactgtagacaaatcctccagcacagcctacatggaagtcagcgacctgacagctgaggactctgcggtctattactgtgcaagaggtagcaacccttactactatgctatggactactggggtcaaggaacctcagtcaccgtctcctca

SEQ ID NO: 5 - DNA, 360 bp, synthetic construct
  source 1..360: mol_type = other DNA
caggtgcaactggagcagcctggggctgaagtggtgaagcctggggcttcagtgaaggtgtcctgcaagacttctggctacaccttcaccagcaactggatgcactgggtgaagcagacgcctggaaaaggacttgagtggatcggagagattgatccttctgatagttatactaactacaatcaaaagttcgatggcaaggccaagttgactgtagacaaatcctccagcacagcctacatggaagtcagcgacctgacagctgaggactctgcgacctattactgtgcaagaggtagcaacccttactactatgctatggactactggggtcaaggaacctcagtcaccgtctcctca

SEQ ID NO: 6 - DNA, 360 bp, synthetic construct
  source 1..360: mol_type = other DNA
caggtgcaactggagcagcctggggctgaagtgaagaagcctggggcttcagtgaaggtgtcctgcaaggcttctggctacaccttcaccagcaactggatgcactgggtgagacaggcacctggaaaaggacttgagtggatcggagagattgatccttctgatagttatactaactacaatcaaaagttcgatggcaaggccaagttgactgtagacaaatcctccagcacagcctacatggaagtcagcgacctgacatctgaggactctgcgacctattactgtgcaagaggtagcaacccttactactatgctatggactactggggtcaaggaacctcagtcaccgtctcctca

SEQ ID NO: 7 - DNA, 360 bp, synthetic construct
  source 1..360: mol_type = other DNA
caggtgcaactggagcagcctggggctgaagtggtgaagcctggggcttcagtgaaggtgtcctgcaagacttctggctacaccttcaccagcaactggatgcactgggtgaagcagaggcctggacaaggacttgagtggatcggagagattgatccttctgatagttatactaactacaatcaaaagttcgatggcaaggccaagttgactgtagacaaatcctccagcacagcctacatggaagtcagcgacctgacatctgaggactctgcgacctattactgtgcaagaggtagcaacccttactactatgctatggactactggggtcaaggaacctcagtcaccgtctcctca

SEQ ID NO: 8 - DNA, 311 bp, synthetic construct
  source 1..311: mol_type = other DNA
caaattgttctcacccagtctccagcaatcatgtctgcatctccaggggagaaggtcaccatgacctgcagtgccagctcaggtgtcaactacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 9 - DNA, 311 bp, synthetic construct
  source 1..311: mol_type = other DNA
caaattgctctcacccaggagccagcaatcatgtctgcatctccaggggagaaggtcaccatgacctgcagtgccagctcaggtgtcaactacatgcactggtatcagcagaagccaggcacctcccccaaaagatggatttatgacacatccaaactggattctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 10 - DNA, 311 bp, synthetic construct
  source 1..311: mol_type = other DNA
gacattgttctcacccagtctccagcaactttgtctgcatctccaggggagaaggtcaccatgacctgtagtgccagctcaggtgtcaactacatgcactggtatcagcagaagccaggcacctcccccaaaagatggatttatgacacatccaaactggattctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 11 - DNA, 311 bp, synthetic construct
  source 1..311: mol_type = other DNA
caaattgttctcacccagtctccagcaactttgtctgcatctccaggggagaaggctaccatgacctgcagtgccagctcaggtgctaactacatgcactggtaccagcagaagccaggcacctcccccaaaagatggatttatgacacatccaaactggcttctggagtccctgctcgcttcagtggcagtgggtctgggacctcttattctctcacaatcgagagcatggaggctgaagatgctgccacttattactgccatcagcgaggtagttacacgttcggaggggggaccaagctggaaataaa

SEQ ID NO: 12 - DNA, 310 bp, synthetic construct
  source 1..310: mol_type = other DNA
caaattgttctcacccagtctccagcaactttgtctgcatctccaggggagaaggctacatgacctgtagtgccagctcaggtgtcaactacatgcactggtaccagcagaagccaggcacctcccccaaaagatggatttatgacacagacaaaacggcttctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 13 - protein, 120 aa, synthetic construct
QVQLQQPGAEVVKPGASVRLSCKTSGYTFTSNWMHWVKQRPGQGLEWIGEIDPSDSYTNYNQKFKGKAKLTVDKSSSTAYMEVSSLTSEDSAVYYCARGSNPYYYAMDYWGQGTSVTVSS

SEQ ID NO: 14 - protein, 120 aa, synthetic construct
QVQLQQPGAEVVKPGASVRLSCETSGYTFTSNWMHWVKQRPDQGLEWIGEIDPSDSYTNYNQKFKGKAELTVDKSSSTAYMEVSDLTSEDSAVYYCARGSNPYYYAMDYWGQGTSVTVSS

SEQ ID NO: 15 - protein, 120 aa, synthetic construct
QVQLQQPGAEVVKPGASVRLSCKTSGYTFTSNWMHWVKQRPGQGLEWIGEIDPSDSYTNYNQKFKGKAELTVDKSSSTAYMEVSDLTSEDSAVYYCARGSNPYYYAMDYWGQGTSVTVSS

SEQ ID NO: 16 - protein, 120 aa, synthetic construct
QVQLEQPGAEVKKPGASVKVSCKASGYTFTSNWMHWVKQRPGQGLEWIGEIDPSDSYTNYNQKFKGKAKLTVDKSSSTAYMEVSDLTAEDSAVYYCARGSNPYYYAMDYWGQGTSVTVSS

SEQ ID NO: 17 - protein, 120 aa, synthetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 18 - protein, 120 aa, synthetic construct
QVQLEQPGAEVKKPGASVKVSCKASGYTFTSNWMHWVRQAPGKGLEWIGEIDPSDSYTNYNQKFDGKAKLTVDKSSSTAYMEVSDLTSEDSATYYCARGSNPYYYAMDYWGQGTSVTVSS

SEQ ID NO: 19 - protein, 120 aa, synthetic construct
QVQLEQPGAEVVKPGASVKVSCKTSGYTFTSNWMHWVKQRPGQGLEWIGEIDPSDSYTNYNQKFDGKAKLTVDKSSSTAYMEVSDLTSEDSATYYCARGSNPYYYAMDYWGQGTSVTVSS

SEQ ID NO: 20 - protein, 120 aa, synthetic construct
QVQLQQPGAEVKKPGASVRLSCETSGYTFTSNWMHWVKQRPDQGLEWIGEIDPSDSYTNYNQKFDGKAELTVDKSSSTAYMEVSDLTSEDSAVYYCARGSNPYYYAMDYWGQGTSVTVSS

SEQ ID NO: 21 - protein, 120 aa, synthetic construct
QVQLEQPGAEVKKPGASVKVSCKASGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 22 - protein, 29 aa, synthetic construct
QVQLXQPGAEVXKPGASVXXSCXXSGYTF

SEQ ID NO: 23 - protein, 14 aa, synthetic construct
WVXQXPXXGLEWIG

SEQ ID NO: 24 - protein, 38 aa, synthetic construct
YNQKFXGKAXLTVDKSSSTAYMEVSXLTXEDSAXYYCA

SEQ ID NO: 25 - protein, 104 aa, synthetic construct
QIVLTQSPAIMSASPGEKVTMTCSASSGVNYMHWYQQKPGTSPKRWIYDTSKLASGVPARFSGSGSGTSYSLTISSMEAEDAATYYCHQRGSYTFGGGTKLEIK

SEQ ID NO: 26 - protein, 104 aa, synthetic construct
QIALTQEPAIMSASPGEKVTMTCSASSGVNYMHWYQQKPGTSPKRWIYDTSKLDSGVPARFSGSGSGTSYSLTISSMEAEDAATYYCHQRGSYTFGGGTKLEIK

SEQ ID NO: 27 - protein, 104 aa, synthetic construct
DIVLTQSPATLSASPGEKVTMTCSASSGVNYMHWYQQKPGTSPKRWIYDTSKLDSGVPARFSGSGSGTSYSLTISSMEAEDAATYYCHQRGSYTFGGGTKLEIK

SEQ ID NO: 28 - protein, 104 aa, synthetic construct
QIVLTQSPATLSASPGEKATMTCSASSGANYMHWYQQKPGTSPKRWIYDTSKLASGVPARFSGSGSGTSYSLTIESMEAEDAATYYCHQRGSYTFGGGTKLEIK

SEQ ID NO: 29 - protein, 104 aa, synthetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIK

SEQ ID NO: 30 - protein, 104 aa, synthetic construct
QIALTQEPAIMSASPGEKATMTCSASSGVNYMHWYQQKPGTSPKRWIYDTSKLDSGVPARFSGSGSGTSYSLTIESMEAEDAATYYCHQRGSYTFGGGTKLEIK

SEQ ID NO: 31 - protein, 104 aa, synthetic construct
QIVLTQSPATLSASPGEKATMTCSASSGANYMHWYQQKPGTSPKRWIYDTDKTASGVPARFSGSGSGTSYSLTIESMEAEDAATYYCHQRGSYTFGGGTKLEIK

SEQ ID NO: 32 - protein, 23 aa, synthetic construct
XIXLTQXPAXXSASPGEKXTMTC

SEQ ID NO: 33 - skipped

SEQ ID NO: 34 - protein, 120 aa, synthetic construct; identical to SEQ ID NO: 13

SEQ ID NO: 35 - protein, 104 aa, synthetic construct; identical to SEQ ID NO: 25

SEQ ID NO: 36 - protein, 9 aa, synthetic construct
VRLSCKTSG

SEQ ID NO: 37 - protein, 9 aa, synthetic construct
WVKQRPGQG

SEQ ID NO: 38 - protein, 9 aa, synthetic construct
YNQKFKGKA

SEQ ID NO: 39 - protein, 9 aa, synthetic construct
FKGKAKLTV

SEQ ID NO: 40 - protein, 9 aa, synthetic construct
YMEVSSLTS

SEQ ID NO: 41 - protein, 9 aa, synthetic construct
VYYCARGSN

SEQ ID NO: 42 - protein, 9 aa, synthetic construct
IVLTQSPAI

SEQ ID NO: 43 - protein, 9 aa, synthetic construct
VLTQSPAIM

SEQ ID NO: 44 - protein, 9 aa, synthetic construct
VTMTCSASS

SEQ ID NO: 45 - protein, 9 aa, synthetic construct
VNYMHWYQQ

SEQ ID NO: 46 - protein, 9 aa, synthetic construct
WIYDTSKLA

SEQ ID NO: 47 - protein, 9 aa, synthetic construct
IYDTSKLAS

SEQ ID NO: 48 - protein, 13 aa, synthetic construct
CGTAAGTGGATCC

SEQ ID NO: 49 - protein, 12 aa, synthetic construct
GCTAGCTCCAGC

SEQ ID NO: 50 - protein, 450 aa, synthetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 450 aa, synthetic construct; identical to SEQ ID NO: 50

SEQ ID NO: 52 - protein, 211 aa, synthetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 211 aa, synthetic construct; identical to SEQ ID NO: 52

SEQ ID NO: 54 - protein, 450 aa, synthetic construct; identical to SEQ ID NO: 50

SEQ ID NO: 55 - protein, 211 aa, synthetic construct; identical to SEQ ID NO: 52